(19) United States
(12) Patent Application Publication
(10) Pub. No.: US 2025/0188118 A1
(43) Pub. Date: Jun. 12, 2025

(54) LIQUID PHASE PEPTIDE SYNTHESIS METHODS

(71) Applicant: AMGEN INC., Thousand Oaks, CA (US)

(72) Inventors: Kyle BAUCOM, Ventura, CA (US); Joshua GLADFELDER, Thousand Oaks, CA (US); Nolan TURMAN, Thousand Oaks, CA (US)

(73) Assignee: AMGEN INC., Thousand Oaks, CA (US)

(21) Appl. No.: 18/967,870

(22) Filed: Dec. 4, 2024

Related U.S. Application Data
(60) Provisional application No. 63/606,885, filed on Dec. 6, 2023.

(51) Int. Cl. C07K 1/12; C07K 1/06; C07K 1/08; C07K 1/10

(52) U.S. Cl. CPC C07K 1/122; C07K 1/061; C07K 1/08; C07K 1/10

(57) ABSTRACT

Disclosed herein are methods of producing peptides, including liquid phase peptide synthesis methods in which one or more cycles of peptide elongation are performed in one pot. In particular, the present disclosure provides liquid phase peptide synthesis methods employing solvent systems comprising 2-methyltetrahydrofuran for condensation reactions, as well as convergent liquid phase peptide assembly methods in which benzyl alcohol anchor groups are selectively removed from an N-protected C-protected peptide by base-catalyzed ester hydrolysis.

### CROSS-REFERENCE TO RELATED APPLICATIONS

This application claims the benefit of priority to U.S. Provisional Patent Application No. 63/606,885, filed Dec. 6, 2023, which is hereby incorporated by reference in its entirety for all purposes.

### FIELD

The present disclosure provides methods of producing peptides, including liquid phase peptide synthesis methods in which one or more cycles of peptide elongation are performed in one pot.

### BACKGROUND

Peptides are becoming increasingly popular as therapeutics, with more than 100 peptide therapeutics marketed worldwide in 2022. Synthetic peptides, which are composed of natural and/or unnatural amino acids and typically have molecular weights in the range of 1 kDa to 5 kDa, are sometimes referred to as “medium molecules”—considerably larger than small molecule drugs but much smaller than large molecule therapeutics such as antibodies and fusion proteins. These medium molecules can be produced at commercial scale using several different approaches, including recombinant production in host cells, solid phase peptide synthesis (SPPS), and liquid phase peptide synthesis (LPPS).

Peptide synthesis using solid phase techniques, in which condensation and deprotection reactions are performed on insoluble resins, is commonly employed because of its speed and minimal labor requirements. However, solid phase peptide synthesis (SPPS) uses large excesses of reagents and significant amounts of solvent in each synthetic step and can result in material batches with sub-optimal purity. By contrast, liquid phase peptide synthesis (LPPS) methods rely on soluble anchor groups or “tags” that enable condensation and deprotection reactions to be carried out in solution. As a result, LPPS methods significantly reduce the required amounts of solvents, amino acids, and coupling agents relative to SPPS methods and can provide material batches of higher purity. While LPPS methods better align with green chemistry principles than SPPS methods due to reduced material consumption, halogenated solvents and other solvents that rank poorly in terms of their health, safety, and environmental effects (e.g., tetrahydrofuran, dichloromethane) are commonly used during LPPS. (See, e.g., Sharma et al., Chem. Rev. 2022, 122, 16, 13516-13546.)

Accordingly, there remains a need in the art for novel liquid phase peptide synthesis methods using greener solvents.

### SUMMARY

Liquid phase peptide synthesis (LPPS) methods provided herein use 2-methyltetrahydrofuran (2-MeTHF), an inexpensive, commercially available solvent that is a green alternative to common LPPS solvents such as dichloromethane (DCM) and tetrahydrofuran (THF).

Disclosed herein is a method of producing a peptide comprising:
(i) condensing an N-terminal amino group of a first C-protected amino acid or a first C-protected peptide with a C-terminal carboxy group of a first N-Fmoc amino acid or a first N-Fmoc peptide in the presence of a first coupling reagent in a first solvent system to obtain a first reaction mixture comprising a first N-Fmoc C-protected peptide, wherein:
   the first solvent system comprises 2-methyltetrahydrofuran (2-MeTHF); and
   a C-terminal carboxy group of the first C-protected amino acid or the first C-protected peptide is protected by an lipophilic molecular anchor group;
(ii) washing the first reaction mixture with a first aqueous solution and separating a first organic layer comprising the first N-Fmoc C-protected peptide;
(iii) removing an N-terminal Fmoc group from the first N-Fmoc C-protected peptide to obtain a second reaction mixture comprising a second C-protected peptide, wherein the removing is performed without an intervening isolation of the first N-Fmoc C-protected peptide from the first organic layer; and
(iv) washing the second reaction mixture with a second aqueous solution and separating a second organic layer comprising the second C-protected peptide.

In some embodiments, the lipophilic molecular anchor group is a lipophilic molecular anchor group described herein. In some embodiments, the lipophilic molecular anchor group is a benzyl alcohol anchor group. In some embodiments, the lipophilic molecular anchor group is derived from 3,4,5-tri(octadecyloxy)benzyl alcohol.

In some embodiments, the first solvent system comprises 2-MeTHF at a concentration in the range of 70% (v/v) to 100% (v/v), e.g., the first solvent system consists of 2-MeTHF. In some embodiments, the first solvent system further comprises dimethylformamide (DMF) or dimethylformamide (DMSO).

In some embodiments, the first coupling reagent is selected from N,N′-dicyclohexylcarbodiimide (DCC), N,N′-diisopropylcarbodiimide (DIC), N-(3-dimethylaminopropyl)-N′-ethylcarbodiimide (EDC), (benzotriazol-1-yloxy)tripyrrolidinophosphonium hexafluorophosphate (PyBOP), [ethyl cyano(hydroxyimino)acetato-O²]tri-1-pyrrolidinylphosphonium hexafluorophosphate (PyOxim), O-(benzotriazol-1-yl)-N,N,N′,N′-tetramethyluronium tetrafluoroborate (TBTU), O-(1H-6-chlorobenzotriazole-1-yl)-1,1,3,3-tetramethyluronium hexafluorophosphate (HCTU), 2-(1H-benzotriazole-1-yl)-1,1,3,3-tetramethyluronium hexafluorophosphate (HBTU), 1-hydroxybenzotriazole (HOBt), ethyl 1-hydroxy-1H-1,2,3-triazole-4-carboxylate (HOCt), 1-hydroxy-7-azabenzotriazole (HOAt), 4-dimethylaminopyridine (DMAP), and combinations of the foregoing.

In some embodiments, the first coupling reagent comprises a condensing agent, such as, e.g., DCC, DIC, EDC, PyBOP, TBTU, HCTU, or HBTU, and an activating agent, such as, e.g., HOBt, HOCt, HOAt, HBTU, HCTU, or DMAP. In some embodiments, the condensing agent is EDC or DIC and the activating agent is HOBt or DMAP.

In some embodiments, the (ii) washing comprises washing the first reaction mixture with a saturated sodium chloride solution (i.e., the first aqueous solution is a saturated sodium chloride solution).

In some embodiments, the (iii) removing comprises removing the N-terminal Fmoc group from the first N-Fmoc C-protected peptide with a first non-nucleophilic organic base in the presence of a first acidic thiol. In some embodiments, the first non-nucleophilic organic base is selected from N,N-diisopropylethylamine, 1,8-diazabicyclo[5.4.0]undec-7-ene (DBU), 1,4-diazabicyclo[2.2.2]octane (DABCO), and 1,5-diazabicyclo[4.3.0]non-5-ene (DBN). In some embodiments, the first non-nucleophilic organic base is DBU. In some embodiments, the first acidic thiol is selected from cysteine, thiomalic acid, and mercaptopropionic acid. In some embodiments, the first acidic thiol is thiomalic acid. In some embodiments, the first non-nucleophilic organic base is DBU and the first acidic thiol is thiomalic acid.

In some embodiments, the (iv) washing comprises washing the second reaction mixture with a second solvent system comprising sodium carbonate and optionally DMF. In some embodiments, the (iv) washing comprises a first wash with a second solvent system comprising sodium carbonate and optionally DMF and one or more additional washes with a saturated sodium chloride solution. In some embodiments, the second solvent system comprises sodium carbonate at a concentration of 10% (v/v). In some embodiments, the second solvent system comprises sodium carbonate and DMF at a 5:1 molar ratio.

In some embodiments, the (iv) washing is performed without an intervening acid neutralization of the second reaction mixture.

In some embodiments, the (i) condensing, the (ii) washing, the (iii) removing, and the (iv) washing are performed in one pot.

Also disclosed herein are peptide chain elongation processes in which the (i) condensing, the (ii) washing, the (iii) removing, and the (iv) washing are repeated for one or more cycles to add one or more amino acids or peptides to a growing peptide chain. In each cycle, the specific conditions for each step in each cycle (e.g., reaction temperature, reaction time, choice and ratio of reagents) can be independently selected. These multiple cycles can also be performed in one pot without, for example, requiring any material to be isolated by precipitation.

For example, in some embodiments in which the (i) condensing, the (ii) washing, the (iii) removing, and the (iv) washing are repeated for one or more cycles, the second C-protected peptide from the (iv) washing in a cycle becomes the first C protected peptide in the (i) condensing of the following cycle; and the first N-Fmoc amino acid or the first N-Fmoc peptide of the (i) condensing of a cycle may be the same as or different from the first N-Fmoc amino acid or the first N Fmoc peptide of the (i) condensing of the preceding cycle.

In some embodiments, the (i) condensing of a given cycle is performed without an intervening isolation of the second C-protected peptide from the second organic layer of the (iv) washing of the preceding cycle.

In liquid phase peptide synthesis processes disclosed herein, a C-protected peptide or a N-Fmoc C-protected peptide can be isolated by antisolvent addition using one or more polar solvents, followed by solid-liquid separation.

Additionally, in liquid phase peptide synthesis processes disclosed herein, a C-protected peptide or a N-Fmoc C-protected peptide can be globally deprotected using an acid, such as, e.g., trifluoroacetic acid (TFA), hydrochloric acid, sulfuric acid, methanesulfonic acid, p-toluenesulfonic acid, or a combination of any of the foregoing, or a fluorine-substituted alcohol, such as, e.g., trifluoroethanol (TFE) or hexafluoroisopropanol (HFIP). Global deprotection methods can remove the lipophilic molecular anchor group, as well as any side chain protecting groups on the peptide.

Alternatively, in liquid phase peptide synthesis processes disclosed herein, a benzyl alcohol anchor group, such as an anchor group derived from 3,4,5-tri(octadecyloxy)benzyl alcohol, can be selectively removed from a C-protected peptide or a N-Fmoc C-protected peptide by base-catalyzed ester hydrolysis (i.e., the anchor group may be removed without removing one or more side chain protecting groups, which may be advantageous when, e.g., convergent assembly of one or more peptides produced by a process disclosed herein into a longer peptide is contemplated). In some embodiments, the base used for base-catalyzed ester hydrolysis is selected from hydroxides soluble in an organic solvent. In some embodiments, the base used for base-catalyzed ester hydrolysis is selected from sodium hydroxide, potassium hydroxide, lithium hydroxide, barium hydroxide, and tetrabutylammonium hydroxide. In some embodiments, the base used for base-catalyzed ester hydrolysis is sodium hydroxide. In some embodiments, the base used for base-catalyzed ester hydrolysis is potassium hydroxide. In some embodiments, the base used for base-catalyzed ester hydrolysis is lithium hydroxide. In some embodiments, the base used for base-catalyzed ester hydrolysis is barium hydroxide. In some embodiments, the base used for base-catalyzed ester hydrolysis is tetrabutylammonium hydroxide.

Also disclosed herein are convergent peptide assembly methods in which C-protected peptides (also referred to as C-protected fragments), e.g., C-protected peptides of 20 or fewer amino acids (e.g., 10 or fewer amino acids), produced by a process described herein, are iteratively coupled to obtain a desired peptide.

In some embodiments, the present disclosure provides a method of producing a peptide comprising:
(i) condensing an N-terminal amino group of a first C-protected peptide with a C-terminal carboxy group of a first N-Boc amino acid or a first N-Boc peptide to obtain a first N-Boc C-protected peptide, wherein a C-terminal carboxy group of the first C-protected peptide is protected by a first benzyl alcohol anchor group;
(ii) removing the first benzyl alcohol anchor group from the first N-Boc C-protected peptide to obtain a second N-Boc peptide;
(iii) condensing a C-terminal carboxy group of the second N-Boc peptide with an N-terminal amino group of a second C-protected peptide to obtain a second N-Boc C-protected peptide, wherein a C-terminal carboxy group of the second C-protected peptide is protected by a second benzyl alcohol anchor group; and
(iv) removing the second benzyl alcohol anchor group from the second N-Boc C-protected peptide to obtain a third N-Boc peptide.

In some embodiments, the first benzyl alcohol anchor group is derived from 3,4,5-tri(octadecyloxy)benzyl alcohol. In some embodiments, the second benzyl alcohol anchor group is derived from 3,4,5-tri(octadecyloxy)benzyl alcohol. In some embodiments, the first benzyl alcohol anchor group and the second benzyl alcohol anchor group are each derived from 3,4,5-tri(octadecyloxy)benzyl alcohol.

In some embodiments, the first C-protected peptide and/or the second C-protected peptide is produced according to a liquid phase peptide synthesis process described herein.

In some embodiments, base-catalyzed ester hydrolysis is used to remove the first benzyl alcohol anchor group from the first N-Boc C-protected peptide and/or to remove the second benzyl alcohol anchor group from the second N-Boc C-protected peptide without removing the side chain protecting groups on the first and/or second N-Boc C-protected peptide. In some embodiments, the base used for base-catalyzed ester hydrolysis is selected from sodium hydroxide, potassium hydroxide, lithium hydroxide, barium hydroxide, and tetrabutylammonium hydroxide. In some embodiments, the base used for base-catalyzed ester hydrolysis is lithium hydroxide. Additionally, in some embodiments, the base-catalyzed ester hydrolysis is performed in a solvent system comprising water and THF, optionally in a 1:4 volumetric ratio.

In addition, in some embodiments, between the (ii) removing and the (iii) condensing, the method optionally further comprises:
(iia) acidifying and diluting the second reaction mixture comprising the second N-Boc peptide;
(iib) separating a second organic layer comprising the second N-Boc peptide and concentrating the second organic layer to obtain a first concentrated mixture comprising the second N-Boc peptide;
(iic) dissolving the first concentrated mixture in a first ethereal solvent, precipitating the first benzyl alcohol anchor group in a second polar solvent system, and removing the first benzyl alcohol anchor group by solid-liquid separation to obtain a first filtrate comprising the second N-Boc peptide; and
(iid) concentrating the first filtrate to isolate the second N-Boc peptide.

The (iii) condensing and (iv) removing may be repeated for one or more cycles to couple additional C-protected fragments to produce a longer peptide. For example, in some embodiments disclosed herein, multiple cycles of convergent peptide assembly can be performed by repeating the general steps of condensation (step (iii)) and benzyl alcohol anchor group removal (step (iv)), where the specific conditions for each step in each cycle (e.g., reaction temperature, reaction time, choice and ratio of reagents) can be independently selected. In some embodiments, between the (iv) removing of one cycle and the (iii) condensing of the next cycle, an N-Boc peptide may be isolated by steps analogous to steps (iia), (iib), (iic), and (iid) above.

Following the final condensation step, the final N-Boc C-protected peptide can be globally deprotected by a variety of means to obtain the assembled peptide. In some embodiments, the final N-Boc C-protected peptide is globally deprotected using an acid (such as, e.g., TFA). In some embodiments, the final N-Boc C-protected peptide is globally deprotected using a fluorine-substituted alcohol (such as, e.g., trifluoroethanol (TFE) or hexafluoroisopropanol (HFIP)).

Example embodiments of the present disclosure include, but are not limited to, the following:
E1. A method of producing a peptide comprising:
   (i) condensing an N-terminal amino group of a first C-protected amino acid or a first C-protected peptide with a C-terminal carboxy group of a first N-Fmoc amino acid or a first N-Fmoc peptide in the presence of a first coupling reagent in a first solvent system to obtain a first reaction mixture comprising a first N-Fmoc C-protected peptide, wherein:
      the first solvent system comprises 2-methyltetrahydrofuran (2-MeTHF); and
      a C-terminal carboxy group of the first C-protected amino acid or the first C-protected peptide is protected by an lipophilic molecular anchor group;
   (ii) washing the first reaction mixture with a first aqueous solution and separating a first organic layer comprising the first N-Fmoc C-protected peptide;
   (iii) removing an N-terminal Fmoc group from the first N-Fmoc C-protected peptide to obtain a second reaction mixture comprising a second C-protected peptide, wherein the removing is performed without an intervening isolation of the first N-Fmoc C-protected peptide from the first organic layer; and
   (iv) washing the second reaction mixture with a second aqueous solution and separating a second organic layer comprising the second C-protected peptide.
E2. The method of E1, wherein the lipophilic molecular anchor group is derived from a compound selected from:
3,4,5-tri(octadecyloxy)benzyl alcohol;
2,4-di(docosyloxy)benzyl alcohol;
4-methoxy-2-[3′,4′,5′-tri(octadecyloxy)benzyloxy]benzyl alcohol;
4-methoxy-2-[3′,4′,5′-tri(octadecyloxy)cyclohexylmethyloxy]benzyl alcohol;
2-methoxy-4-[3′,4′,5′-tri(octadecyloxy)cyclohexylmethyloxy]benzyl alcohol;
4-[3′,4′,5′-tri(octadecyloxy)cyclohexylmethyloxy]benzyl alcohol;
3,5-dimethoxy-4-[3′,4′,5′-tri(octadecyloxy)cyclohexylmethyloxy]benzyl alcohol;
2,4-di(dodecyloxy)benzyl alcohol;
3,4,5-tri(octadecyloxy)benzylamine;
2,4-bisoctadecyloxyphenylmethanol;
2,4-bis-(2-decyl-tetradecyloxy)-phenylmethanol;
2,3,4-trioctadecanoxybenzhydrol;
[phenyl(2,3,4-trioctadecanoxyphenyl)methyl]amine;
4,4′-didocosoxybenzhydrol;
di(4-docosoxyphenyl)methylamine;
4,4-di(12-docosoxydodecyloxy)benzhydrol;
amino-bis[4-(12-docosoxydodecyloxy)phenyl]methane;
N-benzyl-[bis(4-docosyloxyphenyl)]methylamine;
(4-methoxy-phenyl)-[4-(3,4,5-tris-octadecyloxy-cyclohexylmethoxy)-phenyl]-methanol;
{(4-methoxy-phenyl)-[4-(3,4,5-tris-octadecyloxy-cyclohexylmethoxy)-phenyl]-methyl}-amine;
[bis-(4-docosoxy-phenyl)-methyl]-amine;
4-(12′-docosyloxy-1′-dodecyloxy)benzyl alcohol;
4-(12′-docosyloxy-1′-dodecyloxy)-2-methoxybenzyl alcohol;
4-(12′-docosyloxy-1′-dodecyloxy)-2-methoxybenzylamine;
2-(12′-docosyloxy-1′-dodecyloxy)-4-methoxybenzyl alcohol;
2-(12′-docosyloxy-1′-dodecyloxy)-4-methoxybenzylamine;
2-docosyloxy-4-methoxybenzyl alcohol;
4-methoxy-2-[3′,4′,5′-tris(octadecyloxy)benzyloxy)benzyl alcohol;
2-[3′,5′-di(docosyloxy)benzyloxy]-4-methoxybenzyl alcohol;
2-methoxy-4-[2′,2′,2′-tris(octadecyloxymethyl)ethoxy)benzyl alcohol;
2-methoxy-4-[2′,2′,2′-tris(octadecyloxymethyl)ethoxy]benzylamine;
4-methoxy-2-[3′,4′,5′-tris(octadecyloxy)cyclohexylmethyloxy]benzyl alcohol;
2-methoxy-4-[3′,4′,5′-tris(octadecyloxy)cyclohexylmethyloxy]benzyl alcohol;
4-[3′,4′,5′-tris(octadecyloxy)cyclohexylmethyloxy]benzyl alcohol;
3,5-dimethoxy-4-[3′,4′,5′-tris(octadecyloxy)cyclohexylmethyloxy]benzyl alcohol;
N-(4-hydroxymethyl-3-methoxyphenyl) 3,4,5-tris(octadecyloxy)cyclohexylcarboxamide;
N-(5-hydroxymethyl-2-methoxyphenyl) 3,4,5-tris(octadecyloxy)cyclohexylcarboxamide;
N-(4-hydroxymethylphenyl) 3,4,5-tris(octadecyloxy)cyclohexylcarboxamide;
1,22-bis[12-(4-hydroxymethyl-3-methoxyphenoxy)dodecyloxy]docosane; and
1,22-bis[12-(2-hydroxymethyl-5-methoxyphenoxy)dodecyloxy]docosane.
E3. The method of E1, wherein the lipophilic molecular anchor group is derived from a compound selected from

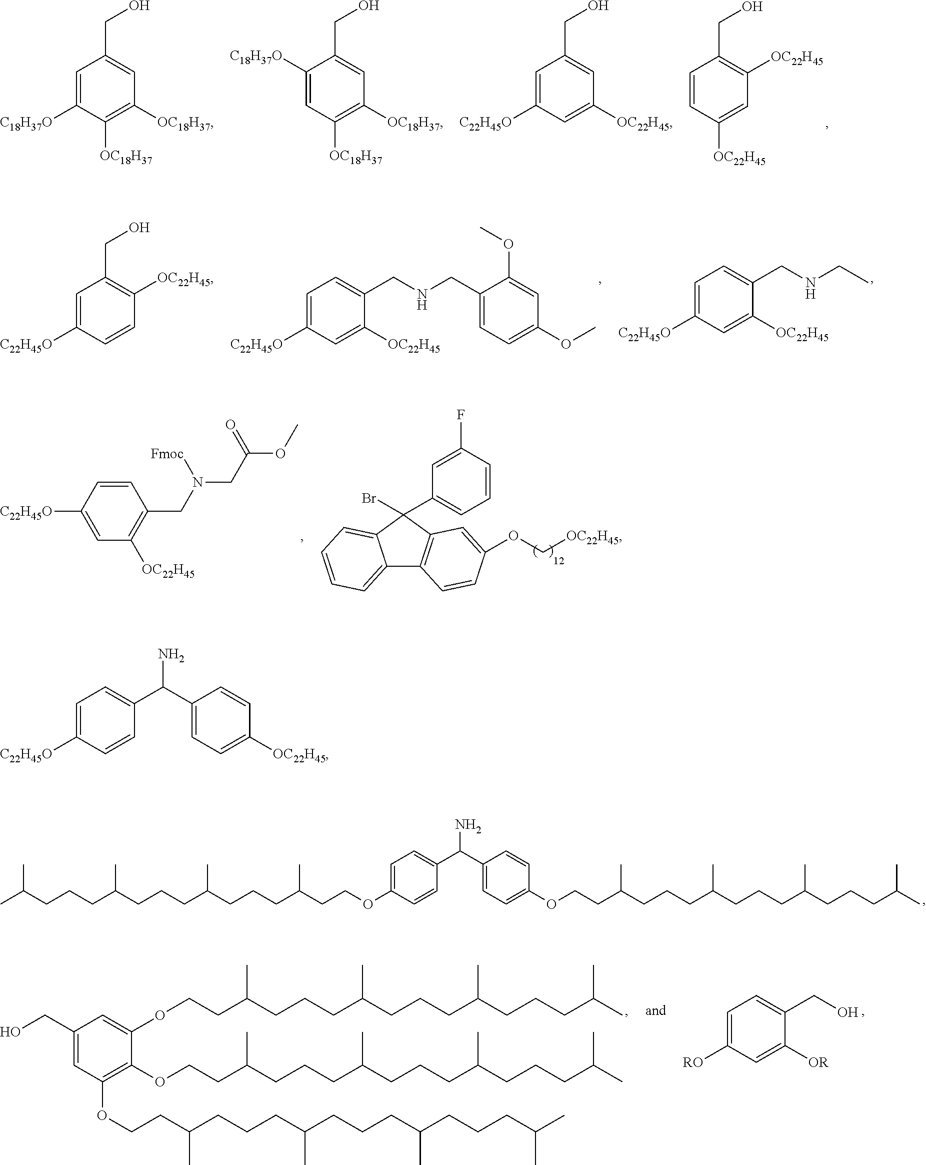

wherein R is

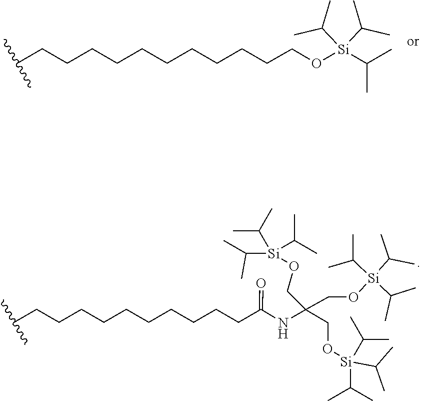 [SMILES: *CCCCCCCCCCCO[Si](C(C)C)(C(C)C)C(C)C ; *CCCCCCCCCCC(=O)NC(CO[Si](C(C)C)(C(C)C)C(C)C)(CO[Si](C(C)C)(C(C)C)C(C)C)CO[Si](C(C)C)(C(C)C)C(C)C]

E4. The method of E1, wherein the lipophilic molecular anchor group is a benzyl alcohol anchor group.
E5. The method of any one of E1-E4, wherein the lipophilic molecular anchor group is derived from 3,4,5-tri(octadecyloxy)benzyl alcohol.
E6. The method of any one of E1-E5, wherein the first solvent system comprises 2-MeTHF at a concentration in the range of 70% (v/v) to 100% (v/v).
E7. The method of any one of E1-E6, wherein the first solvent system further comprises dimethylformamide (DMF).
E8. The method of any one of E1-E7, wherein the first solvent system further comprises dimethylformamide (DMSO).
E9. The method of any one of E1-E6, wherein the first solvent system consists of 2-MeTHF.
E10. The method of any one of E1-E9, wherein the (ii) washing comprises washing the first reaction mixture with a saturated sodium chloride solution.
E11. The method of any one of E1-E10, wherein the (iv) washing comprises washing the second reaction mixture with a second solvent system comprising sodium carbonate.
E12. The method of any one of E1-E11, wherein the (iv) washing comprises washing the second reaction mixture with a second solvent system comprising sodium carbonate and DMF.
E13. The method of any one of E1-E12, wherein the (iv) washing comprises a first wash with a second solvent system comprising sodium carbonate and one or more additional washes with a saturated sodium chloride solution.
E14. The method of any one of E1-E12, wherein the (iv) washing comprises a first wash with a second solvent system comprising sodium carbonate and DMF and one or more additional washes with a saturated sodium chloride solution.
E15. The method of any one of E11-E14, wherein the second solvent system comprises sodium carbonate at a concentration of 10% (v/v).
E16. The method of any one of E11-E15, wherein the second solvent system comprises sodium carbonate and DMF at a 5:1 molar ratio.
E17. The method of any one of E1-E16, wherein the (iv) washing is performed without an intervening acid neutralization of the second reaction mixture.
E18. The method of any one of E1-E17, wherein the (iii) removing comprises removing the N-terminal Fmoc group from the first N-Fmoc C-protected peptide with a first non-nucleophilic organic base in the presence of a first acidic thiol.
E19. The method of E18, wherein the first non-nucleophilic organic base is selected from N,N-diisopropylethylamine, 1,8-diazabicyclo[5.4.0]undec-7-ene (DBU), 1,4-diazabicyclo[2.2.2]octane (DABCO), and 1,5-diazabicyclo[4.3.0]non-5-ene (DBN).
E20. The method of E18 or E19, wherein the first non-nucleophilic organic base is DBU.
E21. The method of any one of E18-E20, wherein the first acidic thiol is selected from cysteine, thiomalic acid, and mercaptopropionic acid.
E22. The method of any one of E18-E21, wherein the first acidic thiol is thiomalic acid.
E23. The method of any one of E18-E22, wherein the first non-nucleophilic organic base is DBU and the first acidic thiol is thiomalic acid.
E24. The method of any one of E1-E23, wherein the first coupling reagent is selected from N,N′-dicyclohexylcarbodiimide (DCC), N,N′-diisopropylcarbodiimide (DIC), N-(3-dimethylaminopropyl)-N′-ethylcarbodiimide (EDC), (benzotriazol-1-yloxy)tripyrrolidinophosphonium hexafluorophosphate (PyBOP), [ethyl cyano(hydroxyimino)acetato-O²]tri-1-pyrrolidinylphosphonium hexafluorophosphate (PyOxim), O-(benzotriazol-1-yl)-N,N,N′,N′-tetramethyluronium tetrafluoroborate (TBTU), O-(1H-6-chlorobenzotriazole-1-yl)-1,1,3,3-tetramethyluronium hexafluorophosphate (HCTU), 2-(1H-benzotriazole-1-yl)-1,1,3,3-tetramethyluronium hexafluorophosphate (HBTU), 1-hydroxybenzotriazole (HOBt), ethyl 1-hydroxy-1H-1,2,3-triazole-4-carboxylate (HOCt), 1-hydroxy-7-azabenzotriazole (HOAt), 4-dimethylaminopyridine (DMAP), and combinations of the foregoing.
E25. The method of any one of E1-E23, wherein the first coupling reagent comprises a condensing agent and an activating agent.
E26. The method of E25, wherein the condensing agent is EDC or DIC.
E27. The method of E25 or E26, wherein the activating agent is HOBt or DMAP.
E28. The method of any one of E1-E27, wherein the (i) condensing, the (ii) washing, the (iii) removing, and the (iv) washing are performed in one pot.
E29. The method of any one of E1-E28, further comprising repeating the (i) condensing, the (ii) washing, the (iii) removing, and the (iv) washing for one or more cycles, wherein:
   the second C-protected peptide from the (iv) washing in a cycle becomes the first C-protected peptide in the (i) condensing of the following cycle; and
   the first N-Fmoc amino acid or the first N-Fmoc peptide of the (i) condensing of a cycle may be the same as or different from the first N-Fmoc amino acid or the first N-Fmoc peptide of the (i) condensing of the preceding cycle.
E30. The method of E29, wherein the (i) condensing of a cycle is performed without an intervening isolation of the second C-protected peptide from the second organic layer of the (iv) washing of the preceding cycle.
E31. The method of E29 or E30, wherein the one or more cycles are performed in one pot.
E32. The method of any one of E1-E31, further comprising:
   (v) condensing an N-terminal amino group of the second C-protected peptide with a C-terminal carboxy group of a second N-Fmoc amino acid or a second N-Fmoc peptide in the presence of a second coupling reagent to obtain a third reaction mixture comprising a second N-Fmoc C-protected peptide, wherein the condensing is performed without an intervening isolation of the second C-protected peptide from the second organic layer;
   (vi) washing the third reaction mixture with a third aqueous solution and separating a third organic layer comprising the second N-Fmoc C-protected peptide;
   (vii) removing an N-terminal Fmoc group from the second N-Fmoc C-protected peptide to obtain a fourth reaction mixture comprising a third C-protected peptide, wherein the removing is performed without an intervening isolation of the second N-Fmoc C-protected peptide from the third organic layer;
   (viii) precipitating the third C-protected peptide in a polar solvent system; and
   (ix) isolating the third C-protected peptide by solid-liquid separation.
E33. The method of E32, wherein the (v) condensing occurs in a third solvent system comprising 2-MeTHF.
E34. The method of E33, wherein the third solvent system comprises 2-MeTHF at a concentration in the range of 70% (v/v) to 100% (v/v).
E35. The method of E33 or E34, wherein the third solvent system further comprises dimethylformamide (DMF).
E36. The method of any one of E33-E35, wherein the third solvent system further comprises dimethylformamide (DMSO).
E37. The method of E33 or E34, wherein the third solvent system consists of 2-MeTHF.
E38. The method of any one of E32-E37, wherein the (vi) washing comprises washing the third reaction mixture with a saturated sodium chloride solution.
E39. The method of any one of E32-E38, wherein the (vii) removing comprises removing the N-terminal Fmoc group from the second N-Fmoc C-protected peptide with a second non-nucleophilic organic base in the presence of a second acidic thiol.
E40. The method of any one of E32-E39, wherein, during the (viii) precipitating, the temperature is cycled for one or more cycles after introduction of the polar solvent system.
E41. The method of any one of E32-E40, wherein, during the (viii) precipitating, the temperature is cycled between 40° C. and 25° C. for one or more cycles after introduction of the polar solvent system.
E42. The method of E32-E41, further comprising washing the third C-protected peptide after the (ix) isolating.
E43. The method of any one of E32-E42, further comprising removing the lipophilic molecular anchor group from the third C-protected peptide.
E44. The method of E43, wherein the lipophilic molecular anchor group is removed from the third C-protected peptide using an acid.
E45. The method of E43 or E44, wherein the lipophilic molecular anchor group is removed from the third C-protected peptide using an acid at a concentration in the range of 0.1% (w/v) to 5% (w/v).
E46. The method of any one of E43-E45, wherein the lipophilic molecular anchor group is removed from the third C-protected peptide using an acid selected from trifluoroacetic acid (TFA), hydrochloric acid, sulfuric acid, methanesulfonic acid, p toluenesulfonic acid, and combinations of any of the foregoing.
E47. The method of any one of E44-E46, wherein the acid is TFA.
E48. The method of E43, wherein the lipophilic molecular anchor group is removed from the third C-protected peptide using a fluorine-substituted alcohol.
E49. The method of E43 or E48, wherein the lipophilic molecular anchor group is removed from the third C-protected peptide using a fluorine-substituted alcohol at a concentration in the range of 10% (w/v) to 100% (w/v).
E50. The method of E48 or E49, wherein the fluorine-substituted alcohol is trifluoroethanol (TFE) or hexafluoroisopropanol (HFIP).
E51. The method of E43, wherein the lipophilic molecular anchor group is a benzyl alcohol anchor group and the benzyl alcohol anchor group is removed from the third C-protected peptide by base-catalyzed ester hydrolysis.
E52. The method of E51, wherein the base used for base-catalyzed ester hydrolysis is selected from sodium hydroxide, potassium hydroxide, lithium hydroxide, barium hydroxide, and tetrabutylammonium hydroxide.
E53. The method of E51 or E52, wherein the base used for base-catalyzed ester hydrolysis is lithium hydroxide.
E54. The method of any one of E51-E53, wherein the base-catalyzed ester hydrolysis is performed in a solvent system comprising water and THF.
E55. The method of any one of E1-E31, further comprising:
   (x) condensing an N-terminal amino group of the second C-protected peptide with a C-terminal carboxy group of a second N-Fmoc amino acid or a second N-Fmoc peptide in the presence of a second coupling reagent to obtain a third reaction mixture comprising a second N-Fmoc C-protected peptide, wherein the condensing is performed without an intervening isolation of the second C-protected peptide from the second organic layer;
   (xi) precipitating the second N-Fmoc C-protected peptide in a polar solvent system; and
   (xii) isolating the second N-Fmoc C-protected peptide by solid-liquid separation.
E56. The method of E55, wherein the (x) condensing occurs in a fourth solvent system comprising 2-MeTHF.
E57. The method of E56, wherein the fourth solvent system comprises 2-MeTHF at a concentration in the range of 70% (v/v) to 100% (v/v).
E58. The method of E56 or E57, wherein the fourth solvent system further comprises dimethylformamide (DMF).
E59. The method of any one of E56-E58, wherein the fourth solvent system further comprises dimethylformamide (DMSO).
E60. The method of E56 or E57, wherein the fourth solvent system consists of 2-MeTHF.
E61. The method of any one of E55-E60, wherein, during the (xi) precipitating, the temperature is cycled for one or more cycles after introduction of the polar solvent system.
E62. The method of any one of E55-E61, wherein, during the (xi) precipitating, the temperature is cycled between 40° C. and 25° C. for one or more cycles after introduction of the polar solvent system.
E63. The method of E55-E62, further comprising washing the second N-Fmoc C-protected peptide after the (xii) isolating.
E64. The method of E55-E63, further comprising removing the lipophilic molecular anchor group from the second N-Fmoc C-protected peptide.
E65. The method of E64, wherein the lipophilic molecular anchor group is removed from the second N-Fmoc C-protected peptide using an acid.
E66. The method of E64 or E65, wherein the lipophilic molecular anchor group is removed from the second N-Fmoc C-protected peptide using an acid at a concentration in the range of 0.1% (w/v) to 5% (w/v).
E67. The method of any one of E64-E66, wherein the lipophilic molecular anchor group is removed from the second N-Fmoc C-protected peptide using an acid selected from trifluoroacetic acid (TFA), hydrochloric acid, sulfuric acid, methanesulfonic acid, p toluenesulfonic acid, and combinations of any of the foregoing.
E68. The method of any one of E65-E67, wherein the acid is TFA.
E69. The method of E64, wherein the lipophilic molecular anchor group is removed from the second N-Fmoc C-protected peptide using a fluorine-substituted alcohol.
E70. The method of E64 or E69, wherein the lipophilic molecular anchor group is removed from the second N-Fmoc C-protected peptide using a fluorine-substituted alcohol at a concentration in the range of 10% (w/v) to 100% (w/v).
E71. The method of E69 or E70, wherein the fluorine-substituted alcohol is trifluoroethanol (TFE) or hexafluoroisopropanol (HFIP).
E72. The method of E64, wherein the lipophilic molecular anchor group is a benzyl alcohol anchor group and the benzyl alcohol anchor group is removed from the third C-protected peptide by base-catalyzed ester hydrolysis.
E73. The method of E72, wherein the base used for base-catalyzed ester hydrolysis is selected from sodium hydroxide, potassium hydroxide, lithium hydroxide, barium hydroxide, and tetrabutylammonium hydroxide.
E74. The method of E72 or E73, wherein the base used for base-catalyzed ester hydrolysis is lithium hydroxide.
E75. The method of any one of E72-E74, wherein the base-catalyzed ester hydrolysis is performed in a solvent system comprising water and THF.
E76. A method of producing a peptide comprising:
   (i) condensing an N-terminal amino group of a first C-protected peptide with a C-terminal carboxy group of a first N-Boc amino acid or a first N-Boc peptide to obtain a first N-Boc C-protected peptide, wherein a C-terminal carboxy group of the first C-protected peptide is protected by a first benzyl alcohol anchor group;
   (ii) removing the first benzyl alcohol anchor group from the first N-Boc C-protected peptide to obtain a second N-Boc peptide;
   (iii) condensing a C-terminal carboxy group of the second N-Boc peptide with an N-terminal amino group of a second C-protected peptide to obtain a second N-Boc C-protected peptide, wherein a C-terminal carboxy group of the second C-protected peptide is protected by a second benzyl alcohol anchor group; and
   (iv) removing the second benzyl alcohol anchor group from the second N-Boc C-protected peptide to obtain a third N-Boc peptide.
E77. The method of E76, wherein the first benzyl alcohol anchor group is derived from 3,4,5-tri(octadecyloxy)benzyl alcohol.
E78. The method of E76 or E77, wherein the second benzyl alcohol anchor group is derived from 3,4,5-tri(octadecyloxy)benzyl alcohol.
E79. The method of any one of E76-E78, wherein the first C-protected peptide is produced according to a method of any one of E1-E54.
E80. The method of any one of E76-E79, wherein the second C-protected peptide is produced according to a method of any one of E1-E54.
E81. The method of any one of E76-E80, wherein the (i) condensing comprises condensing the N-terminal amino group of the first C protected peptide with the C-terminal carboxy group of the first N-Boc amino acid or the first N-Boc peptide in the presence of a first coupling reagent in a first solvent system to obtain a first reaction mixture comprising the first N-Boc C protected peptide.
E82. The method of E81, wherein the first coupling reagent is selected from N,N′-dicyclohexylcarbodiimide (DCC), N,N′-diisopropylcarbodiimide (DIC), N-(3-dimethylaminopropyl)-N′-ethylcarbodiimide (EDC), (benzotriazol-1-yloxy)tripyrrolidinophosphonium hexafluorophosphate (PyBOP), [ethyl cyano(hydroxyimino)acetato-O²]tri-1-pyrrolidinylphosphonium hexafluorophosphate (PyOxim), O-(benzotriazol-1-yl)-N,N,N′,N′-tetramethyluronium tetrafluoroborate (TBTU), O-(1H-6-chlorobenzotriazole-1-yl)-1,1,3,3-tetramethyluronium hexafluorophosphate (HCTU), 2-(1H-benzotriazole-1-yl)-1,1,3,3-tetramethyluronium hexafluorophosphate (HBTU), 1-hydroxybenzotriazole (HOBt), ethyl 1-hydroxy-1H-1,2,3-triazole-4-carboxylate (HOCt), 1-hydroxy-7-azabenzotriazole (HOAt), 4-dimethylaminopyridine (DMAP), and combinations of the foregoing.
E83. The method of E81 or E82, wherein the first coupling reagent comprises a condensing agent and an activating agent.
E84. The method of E83, wherein the condensing agent is EDC or DIC.
E85. The method of E83 or E84, wherein the activating agent is HOBt or DMAP.
E86. The method of any one of E76-E81, wherein, between the (i) condensing and the (ii) removing, the method further comprises (ia) washing the first reaction mixture with a first aqueous solution and separating a first organic layer comprising the first N-Boc C-protected peptide.
E87. The method of E86, wherein the first aqueous solution is a brine solution.
E88. The method of any one of E76-E81, wherein, between the (i) condensing and the (ii) removing, the method further comprises (ia′) precipitating the first N-Boc C-protected peptide in a first polar solvent system and isolating the first N-Boc C-protected peptide by solid-liquid separation.
E89. The method of any one of E76-E89, wherein the (ii) removing comprises removing the first benzyl alcohol anchor group from the first N-Boc C-protected peptide by base-catalyzed ester hydrolysis to obtain a second reaction mixture comprising the second N-Boc peptide.
E90. The method of E89, wherein the base used for base-catalyzed ester hydrolysis is selected from sodium hydroxide, potassium hydroxide, lithium hydroxide, barium hydroxide, and tetrabutylammonium hydroxide.
E91. The method of E89 or E90, wherein the base used for base-catalyzed ester hydrolysis is lithium hydroxide.
E92. The method of any one of E89-E91, wherein the base-catalyzed ester hydrolysis is performed in a solvent system comprising water and THF, optionally in a 1:4 volumetric ratio.
E93. The method of any one of E89-E92, wherein, between the (ii) removing and the (iii) condensing, the method further comprises:
   (iia) acidifying and diluting the second reaction mixture comprising the second N-Boc peptide;
   (iib) separating a second organic layer comprising the second N-Boc peptide and concentrating the second organic layer to obtain a first concentrated mixture comprising the second N-Boc peptide;
   (iic) dissolving the first concentrated mixture in a first ethereal solvent, precipitating the first benzyl alcohol anchor group in a second polar solvent system, and removing the first benzyl alcohol anchor group by solid-liquid separation to obtain a first filtrate comprising the second N-Boc peptide; and
   (iid) concentrating the first filtrate to isolate the second N-Boc peptide.
E94. The method of any one of E76-E93, wherein the (iii) condensing comprises condensing the C-terminal carboxy group of the second N-Boc peptide with the N-terminal amino group of the second C-protected peptide in the presence of a second coupling reagent in a second solvent system to obtain a third reaction mixture comprising the second N-Boc C-protected peptide.
E95. The method of E94, wherein the second coupling reagent is selected from N,N′-dicyclohexylcarbodiimide (DCC), N,N′-diisopropylcarbodiimide (DIC), N-(3-dimethylaminopropyl)-N′-ethylcarbodiimide (EDC), (benzotriazol-1-yloxy)tripyrrolidinophosphonium hexafluorophosphate (PyBOP), [ethyl cyano(hydroxyimino)acetato-O²]tri-1-pyrrolidinylphosphonium hexafluorophosphate (PyOxim), O-(benzotriazol-1-yl)-N,N,N′,N′-tetramethyluronium tetrafluoroborate (TBTU), O-(1H-6-chlorobenzotriazole-1-yl)-1,1,3,3-tetramethyluronium hexafluorophosphate (HCTU), 2-(1H-benzotriazole-1-yl)-1,1,3,3-tetramethyluronium hexafluorophosphate (HBTU), 1-hydroxybenzotriazole (HOBt), ethyl 1-hydroxy-1H-1,2,3-triazole-4-carboxylate (HOCt), 1-hydroxy-7-azabenzotriazole (HOAt), 4-dimethylaminopyridine (DMAP), and combinations of the foregoing.
E96. The method of E94 or E95, wherein the second coupling reagent comprises a condensing agent and an activating agent.
E97. The method of E96, wherein the condensing agent is EDC or DIC.
E98. The method of E96 or E97, wherein the activating agent is HOBt or DMAP.
E99. The method of any one of E76-E98, wherein, between the (iii) condensing and the (iv) removing, the method further comprises (iiia) washing the third reaction mixture with a second aqueous solution and separating a third organic layer comprising the second N-Boc C protected peptide.
E100. The method of E99, wherein the second aqueous solution is a brine solution.
E101. The method of any one of E76-E98, wherein, between the (iii) condensing and the (iv) removing, the method further comprises (iiia′) precipitating the second N-Boc C-protected peptide in a third polar solvent system and isolating the second N-Boc C-protected peptide by solid-liquid separation.
E102. The method of any one of E76-E101, wherein the (iv) removing comprises removing the second benzyl alcohol anchor group from the second N-Boc C-protected peptide by base-catalyzed ester hydrolysis to obtain a fourth reaction mixture comprising the third N-Boc peptide.
E103. The method of E102, wherein the base used for base-catalyzed ester hydrolysis is selected from sodium hydroxide, potassium hydroxide, lithium hydroxide, barium hydroxide, and tetrabutylammonium hydroxide.
E104. The method of E102 or E103, wherein the base used for base-catalyzed ester hydrolysis is lithium hydroxide.
E105. The method of any one of E102-E104, wherein the base-catalyzed ester hydrolysis is performed in a solvent system comprising water and THF, optionally in a 1:4 volumetric ratio.
E106. The method of any one of E76-E105, wherein the method further comprises repeating the (iii) condensing and the (iv) removing for one or more cycles, wherein:
   the third N-Boc peptide from the (iv) removing in a cycle becomes the second N-Boc peptide in the (iii) condensing of the following cycle; and
   the second C-protected peptide of the (iii) condensing of a cycle may be the same as or different from the second C-protected peptide of the (iii) condensing of the preceding cycle.
E107. The method of E106, wherein, between the (iv) removing of a cycle and the (iii) condensing of the following cycle, the method further comprises:
   (iia) acidifying and diluting a fourth reaction mixture comprising the third N-Boc peptide;
   (iib) separating a fourth organic layer comprising the third N-Boc peptide and concentrating the fourth organic layer to obtain a second concentrated mixture comprising the third N-Boc peptide;
   (iic) dissolving the second concentrated mixture in a second ethereal solvent, precipitating the second benzyl alcohol anchor group in a fourth polar solvent system, and removing the second benzyl alcohol anchor group by solid-liquid separation to obtain a second filtrate comprising the third N-Boc peptide; and
   (iid) concentrating the second filtrate to isolate the third N-Boc peptide.
E108. The method of E106 or E107, wherein, following a final (iii) condensing, a final N-Boc C-protected peptide is globally deprotected to obtain a peptide that does not comprise a C-terminal protecting group, an N-terminal protecting group, and/or a side chain protecting group.
E109. The method of E108, wherein the final N-Boc C-protected peptide is globally deprotected using an acid.
E110. The method of E109, wherein the acid is at a concentration in the range of 0.1% (w/v) to 5% (w/v).
E111. The method of E109 or E110, wherein the acid is selected from trifluoroacetic acid (TFA), hydrochloric acid, sulfuric acid, methanesulfonic acid, p toluenesulfonic acid, and combinations of any of the foregoing.
E112. The method of any one of E109-E111, wherein the acid is TFA.
E113. The method of E108, wherein the final N-Boc C-protected peptide is globally deprotected using a fluorine-substituted alcohol.
E114. The method of E113, wherein the fluorine-substituted alcohol is at a concentration in the range of 10% (w/v) to 100% (w/v).
E115. The method of E113 or E114, wherein the fluorine-substituted alcohol is trifluoroethanol (TFE) or hexafluoroisopropanol (HFIP).

### BRIEF DESCRIPTION OF THE DRAWINGS

FIG. 1 is a schematic showing a non-limiting example of a convergent liquid phase peptide synthesis process according to the present disclosure.

### DETAILED DESCRIPTION

### Definitions

The following definitions are provided to assist in understanding the scope of this disclosure. Unless defined otherwise, all technical and scientific terms used herein have the same meaning as is commonly understood by one of ordinary skill in the art to which this disclosure belongs.

As used herein, the term “acidic thiol” refers to a compound represented by the formula HS-L-COOH, wherein L is an optionally substituted C₁₋₈-alkylene group. Non-limiting examples of acidic thiols include cysteine, thiomalic acid, and mercaptopropionic acid.

As used herein, the term “alkyl” refers to a saturated straight chain hydrocarbon or saturated branched chain hydrocarbon containing the indicated number of carbon atoms. For example, C₃alkyl means an alkyl group that has 3 carbon atoms (e.g., n-propyl or isopropyl). For example, a C₁₋₆alkyl refers to an alkyl group having 1 to 6 carbon atoms. Where a range is indicated, all members of that range and all subgroups within that range are envisioned. For example, a C₁₋₆alkyl includes alkyl groups having 1, 2, 3, 4, 5, or 6 carbon atoms (or any combination of the foregoing), as well as all subgroups in the indicated range (e.g., 1-2, 1-3, 1-4, 1-5, 1-6, 2-3, 2-4, 2-5, 2-6, 3-4, 3-5, 3-6, 4-5, 4-6, or 5-6 carbon atoms, or any combination of the foregoing ranges)). A “C₁₋₄ alkyl” includes, for example, methyl, ethyl, n-propyl, isopropyl, n-butyl, isobutyl, sec-butyl, or t-butyl. Nonlimiting examples of alkyl groups include methyl, ethyl, n-propyl, isopropyl, n-butyl, sec-butyl, isobutyl, tert-butyl, n-pentyl, and n-hexyl.

As used herein, the term “alkylene” refers to a divalent saturated, straight or branched hydrocarbon chain diradical containing the indicated number of carbon atoms. For example, C₃alkylene means the alkylene group has 3 carbon atoms. Where a range is indicated, all members of that range and all subgroups within that range are envisioned. For example, C₁₋₆alkylene means an alkylene group having a 1, 2, 3, 4, 5, or 6 carbon atoms, or any combination of the foregoing), as well as all subgroups in the indicated range (e.g., 1-2, 1-3, 1-4, 1-5, 1-6, 2-3, 2-4, 2-5, 2-6, 3-4, 3-5, 3-6, 4-5, 4-6, and 5-6 carbon atoms, or any combination of the foregoing). When the number of carbon atoms in an alkylene group is indicated as “C₀,” then the alkylene group is not present and the recited substituent is directly attached to the rest of the compound. For example, the term C₀₋₆alkylene-OH indicates that the OH group can be directly attached to the compound or through a C₁₋₆alkylene linker. Examples of alkylene groups include methylene (—CH₂—), ethylene (—CH₂CH₂—), n-propylene (—CH₂CH₂CH₂—), isopropylene (—CH(CH₃)CH₂—), 1-butylene (—CH₂CH₂CH₂CH₂—), 1-methylbutylene (—CH(CH₃)CH₂CH₂—), 2-methylbutylene (—CH₂CH(CH₃)CH₂—), and 3-methylbutylene (—CH₂CH₂CH₂(CH₃)—).

As used herein, the terms “alkoxy” and “alkoxyl” are interchangeable and refer to an —O-alkyl group, where the alkyl group is as defined elsewhere herein. For example, a C₃alkoxy group means the alkoxy group has 3 carbon atoms (e.g., OCH₂CH₂CH₃). Where a range is indicated, all members of that range and all subgroups within that range are envisioned. For example, a C₁₋₆alkoxy includes alkoxy groups having 2, 3, 4, 5, or 6 carbon atoms, or any combination of the foregoing, as well as all subgroups in the indicated range (e.g., 2-3, 2-4, 2-5, 2-6, 3-4, 3-5, 3-6, 4-5, 4-6, and 5-6 carbon atoms, or any combination of the foregoing). Nonlimiting examples of alkoxy groups include methoxy, ethoxy, n-propoxy, 1-methylethyloxy (iso-propoxy), n-butoxy, isobutoxy, sec-butoxy, and tert-butoxy.

As used herein, the term “amino” refers to —NH₂.

As used herein, the term “amino acid” refers to a natural or a non-natural amino acid (e.g., 2-aminoisobutyric acid) comprising a terminal amino group and a terminal carboxy group. Amino acid, as used herein, includes amino acids that are not gene-encoded, as well as amino acids that have been modified to include reactive groups or glycosylation sites. Additionally, an amino acid described herein can be a D-isomer or an L-isomer.

As used herein, the term “lipophilic molecular anchor group” refers to a protecting group comprising one or more long aliphatic alkoxy chains (e.g., aliphatic alkoxy chains with 10 or more carbon atoms, such as, e.g., with 18 or more carbon atoms). Lipophilic molecular anchor groups can render an amino acid or a peptide hydrophobic, which can facilitate solution chemistry and workup during liquid phase peptide synthesis. As used herein, a lipophilic molecular anchor group is described as being “derived from” a compound when that compound is condensed with an amino acid or a peptide to protect the C-terminal carboxy group of the amino acid or peptide. Illustratively, a compound comprising an —OH group can be coupled with an amino acid or a peptide via an ester linkage to form a C-protected amino acid or C-protected peptide, respectively. In that case, the lipophilic molecular anchor group on the C-protected amino acid or C-protected peptide would be described as being “derived from” the compound. Similarly, a compound comprising an —NH₂ group can be coupled with an amino acid or a peptide via an amide linkage to form a C-protected amino acid or C-protected peptide, respectively, and the lipophilic molecular anchor group of the C-protected amino acid or C-protected peptide would be considered to be “derived from” the compound.

As used herein, the term “benzyl alcohol anchor group” refers to a lipophilic molecular anchor group derived from a substituted benzyl alcohol.

As used herein, the term “C-protected amino acid” refers to an amino acid in which the C-terminal carboxy group is protected by an anchor group and the N-terminal amino group is not protected.

As used herein, the term “C-protected peptide” refers to a peptide in which the C-terminal carboxy group is protected by an anchor group and the N-terminal amino group is not protected.

As used herein, the term “ether” refers to an oxygen atom bonded to two alkyl or aryl groups (R—O—R).

As used herein, the terms “hydroxy” and “hydroxyl” are interchangeable and refer to a —OH group.

As used herein, the term “N-Boc amino acid” refers to an amino acid in which the N-terminal amino group is protected by a tert-butyloxycarbonyl (Boc) group and the C-terminal carboxy group is not protected.

As used herein, the term “N-Boc peptide” refers to a peptide in which the N-terminal amino group is protected by a tert-butyloxycarbonyl (Boc) group and the C-terminal carboxy group is not protected.

As used herein, the term “N-Fmoc amino acid” refers to an amino acid in which the N-terminal amino group is protected by a 9-fluorenylmethyloxycarbonyl (Fmoc) group and the C-terminal carboxy group is not protected.

As used herein, the term “N-Fmoc peptide” refers to a peptide in which the N-terminal amino group is protected by an Fmoc group and the C-terminal carboxy group is not protected.

As used herein, the term “N-Boc C-protected peptide” refers to a peptide in which the N-terminal amino group is protected by a Boc group and the C-terminal carboxy group is protected by an anchor group.

As used herein, the term “N-Fmoc C-protected amino acid” refers to an amino acid in which the N-terminal amino group is protected by an Fmoc group and the C-terminal carboxy group is protected by an anchor group.

As used herein, the term “N-Fmoc C-protected peptide” refers to a peptide in which the N-terminal amino group is protected by an Fmoc group and the C-terminal carboxy group is protected by an anchor group.

As used herein, the term “N-protected C-protected peptide” refers to a peptide in which the N-terminal amino group is protected by a protecting group (e.g., a Boc group or an Fmoc group) and the C-terminal carboxy group is protected by an anchor group.

As used herein, the term “peptide” refers to a polymer in which the monomers are amino acids that are joined together through amide bonds. The amino acid monomers may be natural or non-natural amino acids (e.g., β-alanine, phenylglycine, homoarginine) and include amino acids that are not gene-encoded, as well as amino acids that have been modified to include reactive groups or glycosylation sites. Additionally, the amino acid monomers may be D-isomers or L-isomers. Peptides disclosed herein may be glycosylated or unglycosylated.

As used herein, the term “protecting group” refers to a removable moiety that modifies a desired functional group to block the desired functional group from reacting in a subsequent chemical reaction. For example, the term “nitrogen protecting group” refers to a removable moiety that modifies a functional group having a nitrogen atom to block the functional group having a nitrogen atom from reacting in a subsequent chemical reaction (e.g., tert-butyloxycarbonyl). Examples of protecting groups are detailed in Greene, T. W., Wuts, P. G, “Protective Groups in Organic Synthesis”, Third Edition, John Wiley & Sons, New York: 1999 (and other editions of the book, such as Wuts, P. G. M. and Greene, T. W. “Greene's Protective Groups in Organic Synthesis,” Fourth Edition, John Wiley & Sons, Hoboken: 2007).

As used herein, the term “substituted” refers to the replacement of one or more hydrogen radicals in a given structure or functional group with the radical of a specified substituent. A substituted structure or functional group may have a substituent at any substitutable position of the structure or functional group. When more than one position in a given structure can be substituted with more than one substituent, the substituent may be either the same or different at each position.

As used herein, the term “thiol” refers to a —SH group.

As used herein, if any variable occurs more than one time in a chemical formula, its definition on each occurrence is independent of its definition at every other occurrence.

### Solvent Systems for Condensation Reactions

2-methyltetrahydrofuran (2-MeTHF) is an inexpensive, commercially available, neoteric, bio-based solvent. 2-MeTHF can be derived from renewable resources (e.g., corn; bagasse) and is commonly regarded as a green solvent. 2-MeTHF forms an azeotrope rich with water and exhibits limited miscibility in water (14 g/100 g at 23° C.), enabling facile separation and drying.

2-MeTHF can be used as a solvent for condensation reactions in the liquid phase peptide synthesis methods provided herein. For example, in certain methods of the present disclosure, an N-terminal amino group of a C-protected amino acid or a C protected peptide can be coupled with a C-terminal carboxy group of a N-Fmoc amino acid or a N-Fmoc peptide using coupling reagents in a 2-MeTHF-containing solvent system to obtain a reaction mixture comprising a N-Fmoc C-protected peptide.

In some embodiments of the present disclosure, the solvent system comprises at least 70% (v/v), at least 71% (v/v), at least 72% (v/v), at least 73% (v/v), at least 74% (v/v), at least 75% (v/v), at least 76% (v/v), at least 77% (v/v), at least 78% (v/v), at least 79% (v/v), at least 80% (v/v), at least 81% (v/v), at least 82% (v/v), at least 83% (v/v), at least 84% (v/v), at least 85% (v/v), at least 86% (v/v), at least 87% (v/v), at least 88% (v/v), at least 89% (v/v), at least 90% (v/v), at least 91% (v/v), at least 92% (v/v), at least 93% (v/v), at least 94% (v/v), at least 95% (v/v), at least 96% (v/v), at least 97% (v/v), at least 98% (v/v), or at least 99% (v/v) 2-MeTHF.

In some embodiments of the present disclosure, the solvent system comprises at least 70% (v/v) 2-MeTHF. In some embodiments, the solvent system comprises at least 75% (v/v) 2-MeTHF. In some embodiments, the solvent system comprises at least 80% (v/v) 2-MeTHF. In some embodiments, the solvent system comprises at least 85% (v/v) 2-MeTHF. In some embodiments, the solvent system comprises at least 90% (v/v) 2-MeTHF. In some embodiments, the solvent system comprises at least 95% (v/v) 2-MeTHF.

In some embodiments of the present disclosure, the solvent system comprises 70% (v/v), 71% (v/v), 72% (v/v), 73% (v/v), 74% (v/v), 75% (v/v), 76% (v/v), 77% (v/v), 78% (v/v), 79% (v/v), 80% (v/v), 81% (v/v), 82% (v/v), 83% (v/v), 84% (v/v), 85% (v/v), 86% (v/v), 87% (v/v), 88% (v/v), 89% (v/v), 90% (v/v), 91% (v/v), 92% (v/v), 93% (v/v), 94% (v/v), 95% (v/v), 96% (v/v), 97% (v/v), 98% (v/v), or 99% (v/v) 2-MeTHF.

In some embodiments of the present disclosure, the solvent system comprises 70% (v/v) 2-MeTHF. In some embodiments, the solvent system comprises 75% (v/v) 2-MeTHF. In some embodiments, the solvent system comprises 80% (v/v) 2-MeTHF. In some embodiments, the solvent system comprises 85% (v/v) 2-MeTHF. In some embodiments, the solvent system comprises 90% (v/v) 2-MeTHF. In some embodiments, the solvent system comprises 95% (v/v) 2-MeTHF. In some embodiments, the solvent system consists of 2-MeTHF.

The solvent system for each condensation reaction can independently further comprise at least one additional solvent (e.g., at least one additional organic solvent). In some embodiments of the present disclosure, the at least one additional solvent is selected from dimethylformamide (DMF) and dimethyl sulfoxide (DMSO). In some embodiments, the at least one additional solvent is DMF. In some embodiments, the at least one additional solvent is DMSO. In some embodiments, the at least one additional solvent is a mixture of DMF and DMSO.

In some embodiments of the present disclosure, the solvent system comprises 2-MeTHF at a concentration in the range of 70% (v/v) to 100% (v/v) and DMSO at a concentration in the range of 0% (v/v) to 30% (v/v), wherein the total concentration of 2-MeTHF and DMSO is less than or equal to 100% (v/v). In some embodiments, the solvent system comprises 2-MeTHF at a concentration in the range of 70% (v/v) to 100% (v/v) and DMSO at a concentration in the range of 0% (v/v) to 30% (v/v), wherein the total concentration of 2-MeTHF and DMSO is 100% (v/v). In some embodiments, the solvent system comprises 2-MeTHF at a concentration in the range of 80% (v/v) to 100% (v/v) and DMSO at a concentration in the range of 0% (v/v) to 20% (v/v), wherein the total concentration of 2-MeTHF and DMSO is less than or equal to 100% (v/v). In some embodiments, the solvent system comprises 2-MeTHF at a concentration in the range of 80% (v/v) to 100% (v/v) and DMSO at a concentration in the range of 0% (v/v) to 20% (v/v), wherein the total concentration of 2-MeTHF and DMSO is 100% (v/v).

In some embodiments, the solvent system comprises 70% (v/v) 2-MeTHF and 30% (v/v) DMSO. In some embodiments, the solvent system comprises 75% (v/v) 2-MeTHF and 25% (v/v) DMSO. In some embodiments, the solvent system comprises 80% (v/v) 2-MeTHF and 20% (v/v) DMSO. In some embodiments, the solvent system comprises 85% (v/v) 2-MeTHF and 15% (v/v) DMSO. In some embodiments, the solvent system comprises 90% (v/v) 2-MeTHF and 10% (v/v) DMSO. In some embodiments, the solvent system comprises 95% (v/v) 2-MeTHF and 5% (v/v) DMSO.

In some embodiments of the present disclosure, the solvent system comprises 2-MeTHF at a concentration in the range of 70% (v/v) to 100% (v/v) and DMF at a concentration in the range of 0% (v/v) to 30% (v/v), wherein the total concentration of 2-MeTHF and DMF is less than or equal to 100% (v/v). In some embodiments, the solvent system comprises 2-MeTHF at a concentration in the range of 70% (v/v) to 100% (v/v) and DMF at a concentration in the range of 0% (v/v) to 30% (v/v), wherein the total concentration of 2-MeTHF and DMF is 100% (v/v). In some embodiments, the solvent system comprises 2-MeTHF at a concentration in the range of 80% (v/v) to 100% (v/v) and DMF at a concentration in the range of 0% (v/v) to 20% (v/v), wherein the total concentration of 2-MeTHF and DMF is less than or equal to 100% (v/v). In some embodiments, the solvent system comprises 2-MeTHF at a concentration in the range of 80% (v/v) to 100% (v/v) and DMF at a concentration in the range of 0% (v/v) to 20% (v/v), wherein the total concentration of 2-MeTHF and DMF is 100% (v/v).

In some embodiments, the solvent system comprises 70% (v/v) 2-MeTHF and 30% (v/v) DMF. In some embodiments, the solvent system comprises 75% (v/v) 2-MeTHF and 25% (v/v) DMF. In some embodiments, the solvent system comprises 80% (v/v) 2-MeTHF and 20% (v/v) DMF. In some embodiments, the solvent system comprises 85% (v/v) 2-MeTHF and 15% (v/v) DMF. In some embodiments, the solvent system comprises 90% (v/v) 2-MeTHF and 10% (v/v) DMF. In some embodiments, the solvent system comprises 95% (v/v) 2-MeTHF and 5% (v/v) DMF.

### Lipophilic Molecular Anchor Groups

Liquid phase peptide synthesis (LPPS) methods, such as those described herein, rely on soluble anchor groups that enable condensation and deprotection reactions to be carried out in solution. The soluble anchor group, which is sometimes also referred to as a soluble tag, confers the growing peptide chain with physicochemical properties that facilitate solution chemistry and workup after each synthetic step. Accordingly, the properties of anchor groups used for LPPS methods must sufficiently differ from the properties of the reagents and byproducts of LPPS to facilitate removal of excess reagents and byproducts by methods such as precipitation, filtration, and extraction.

The LPPS methods disclosed herein can employ lipophilic molecular anchor groups comprising long aliphatic alkoxy chains, which confer hydrophobicity to the growing peptide chain and enable facile isolation by phase separation with aqueous solutions or precipitation in polar solvents such as acetonitrile and methanol.

Lipophilic molecular anchor groups suitable for use in the described methods include, but are not limited to, those described in U.S. Pat. Nos. 8,293,948, 8,633,298, 9,029,504, 9,206,230, and 9,670,121, which are incorporated by reference herein with respect to the identities of the described anchor groups.

In some embodiments, the lipophilic molecular anchor group is soluble in a halogenated solvent. In some embodiments, the lipophilic molecular anchor group is soluble in an ether solvent. In some embodiments, the lipophilic molecular anchor group is insoluble in a polar solvent. In some embodiments, the lipophilic molecular anchor group is soluble in an ethereal solvent and insoluble in a polar solvent.

In some embodiments, the lipophilic molecular anchor group has a molecule weight of at least 300 Da. In some embodiments, the lipophilic molecular anchor group has a molecule weight of at least 400 Da.

Herein, a lipophilic molecular anchor group is described as being derived from a compound when that compound is condensed with an amino acid or a peptide to protect the C-terminal carboxy group of the amino acid or peptide. In some embodiments, the condensation reaction between the compound and the C-terminal carboxy group of the amino acid or peptide is an esterification reaction or an amidation reaction.

For example,

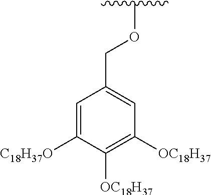

is a lipophilic molecular anchor group derived from

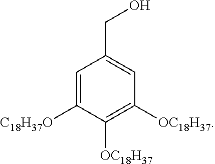

Similarly, as another example,

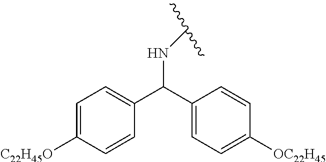

is a lipophilic molecular anchor group derived from

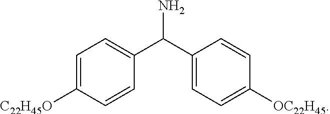

In some embodiments, the lipophilic molecular anchor group is derived from a compound selected from

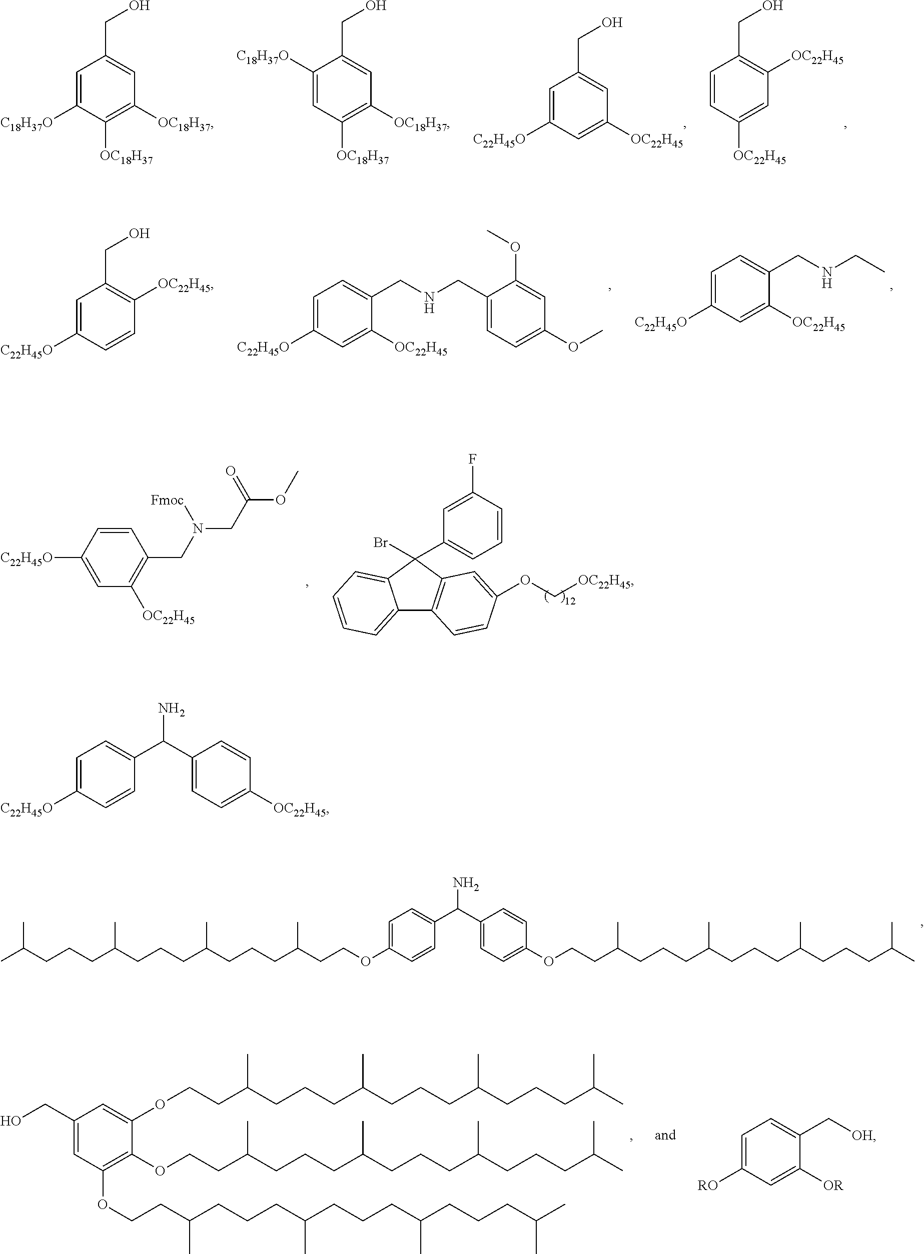

wherein R is

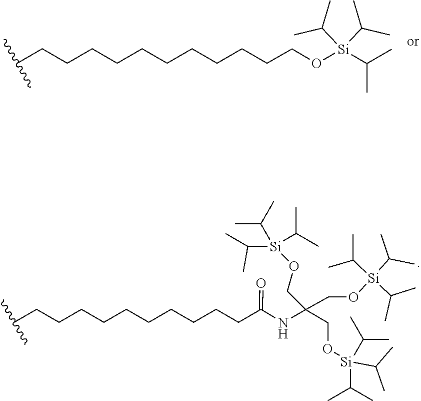 [SMILES: *CCCCCCCCCCCO[Si](C(C)C)(C(C)C)C(C)C ; *CCCCCCCCCCC(=O)NC(CO[Si](C(C)C)(C(C)C)C(C)C)(CO[Si](C(C)C)(C(C)C)C(C)C)CO[Si](C(C)C)(C(C)C)C(C)C]

In some embodiments, the lipophilic molecular anchor group is derived from a compound selected from

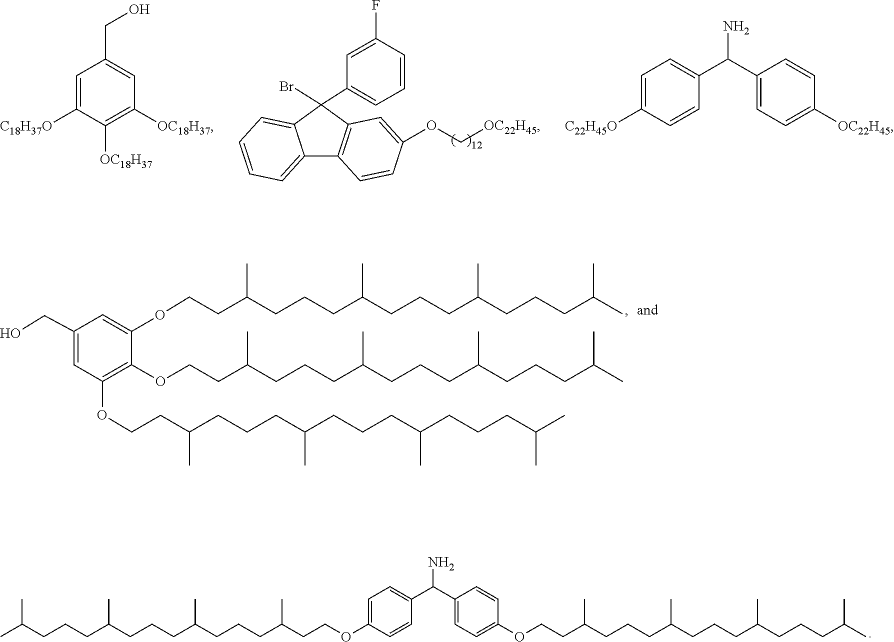

In some embodiments, the lipophilic molecular anchor group is derived from

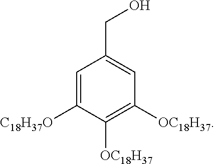

In some embodiments, the lipophilic molecular anchor group is derived from

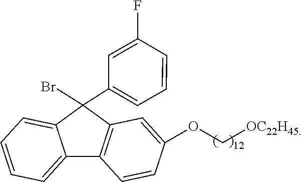

In some embodiments, the lipophilic molecular anchor group is derived from

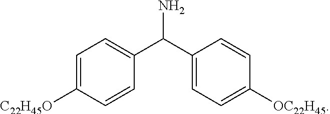

In some embodiments, the lipophilic molecular anchor group is derived from

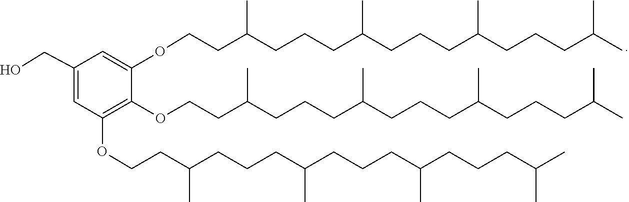 [SMILES: CC(C)CCCC(C)CCCC(C)CCCC(C)CCOc1cc(CO)cc(OCCC(C)CCCC(C)CCCC(C)CCCC(C)C)c1OCCC(C)CCCC(C)CCCC(C)CCCC(C)C]

In some embodiments, the lipophilic molecular anchor group is derived from

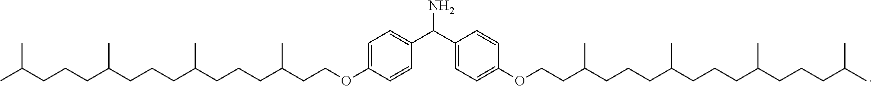 [SMILES: CC(C)CCCC(C)CCCC(C)CCCC(C)CCOc1ccc(C(N)c2ccc(OCCC(C)CCCC(C)CCCC(C)CCCC(C)C)cc2)cc1]

In some embodiments, the lipophilic molecular anchor group is derived from

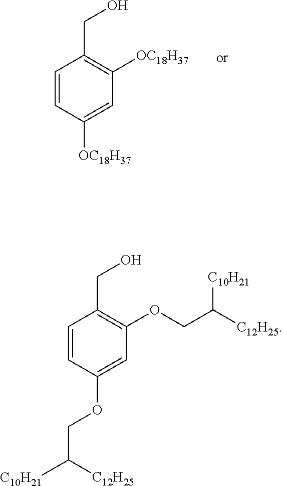

In some embodiments, the lipophilic molecular anchor group is derived from

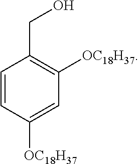

In some embodiments, the lipophilic molecular anchor group is derived from

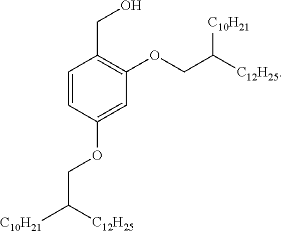

In some embodiments, the lipophilic molecular anchor group is derived from a compound selected from:
3,4,5-tri(octadecyloxy)benzyl alcohol;
2,4-di(docosyloxy)benzyl alcohol;
4-methoxy-2-[3′,4′,5′-tri(octadecyloxy)benzyloxy]benzyl alcohol;
4-methoxy-2-[3′,4′,5′-tri(octadecyloxy)cyclohexylmethyloxy]benzyl alcohol;
2-methoxy-4-[3′,4′,5′-tri(octadecyloxy)cyclohexylmethyloxy]benzyl alcohol;
4-[3′,4′,5′-tri(octadecyloxy)cyclohexylmethyloxy]benzyl alcohol;
3,5-dimethoxy-4-[3′,4′,5′-tri(octadecyloxy)cyclohexylmethyloxy]benzyl alcohol;
2,4-di(dodecyloxy)benzyl alcohol;
3,4,5-tri(octadecyloxy)benzylamine;
2,4-bisoctadecyloxyphenylmethanol;
2,4-bis-(2-decyl-tetradecyloxy)-phenylmethanol;
2,3,4-trioctadecanoxybenzhydrol;
[phenyl(2,3,4-trioctadecanoxyphenyl)methyl]amine;
4,4′-didocosoxybenzhydrol;
di(4-docosoxyphenyl)methylamine;
4,4-di(12-docosoxydodecyloxy)benzhydrol;
amino-bis[4-(12-docosoxydodecyloxy)phenyl]methane;
N-benzyl-[bis(4-docosyloxyphenyl)]methylamine;
(4-methoxy-phenyl)-[4-(3,4,5-tris-octadecyloxy-cyclohexylmethoxy)-phenyl]-methanol;
{(4-methoxy-phenyl)-[4-(3,4,5-tris-octadecyloxy-cyclohexylmethoxy)-phenyl]-methyl}-amine;
[bis-(4-docosoxy-phenyl)-methyl]-amine;
4-(12′-docosyloxy-1′-dodecyloxy)benzyl alcohol;
4-(12′-docosyloxy-1′-dodecyloxy)-2-methoxybenzyl alcohol;
4-(12′-docosyloxy-1′-dodecyloxy)-2-methoxybenzylamine;
2-(12′-docosyloxy-1′-dodecyloxy)-4-methoxybenzyl alcohol;
2-(12′-docosyloxy-1′-dodecyloxy)-4-methoxybenzylamine;
2-docosyloxy-4-methoxybenzyl alcohol;
4-methoxy-2-[3′,4′,5′-tris(octadecyloxy)benzyloxy)benzyl alcohol;
2-[3′,5′-di(docosyloxy)benzyloxy]-4-methoxybenzyl alcohol;
2-methoxy-4-[2′,2′,2′-tris(octadecyloxymethyl)ethoxy)benzyl alcohol;
2-methoxy-4-[2′,2′,2′-tris(octadecyloxymethyl)ethoxy]benzylamine;
4-methoxy-2-[3′,4′,5′-tris(octadecyloxy)cyclohexylmethyloxy]benzyl alcohol;
2-methoxy-4-[3′,4′,5′-tris(octadecyloxy)cyclohexylmethyloxy]benzyl alcohol;
4-[3′,4′,5′-tris(octadecyloxy)cyclohexylmethyloxy]benzyl alcohol;
3,5-dimethoxy-4-[3′,4′,5′-tris(octadecyloxy)cyclohexylmethyloxy]benzyl alcohol;
N-(4-hydroxymethyl-3-methoxyphenyl) 3,4,5-tris(octadecyloxy)cyclohexylcarboxamide;
N-(5-hydroxymethyl-2-methoxyphenyl) 3,4,5-tris(octadecyloxy)cyclohexylcarboxamide;
N-(4-hydroxymethylphenyl) 3,4,5-tris(octadecyloxy)cyclohexylcarboxamide;
1,22-bis[12-(4-hydroxymethyl-3-methoxyphenoxy)dodecyloxy]docosane; and
1,22-bis[12-(2-hydroxymethyl-5-methoxyphenoxy)dodecyloxy]docosane.

In some embodiments, the lipophilic molecular anchor group is derived from 3,4,5-tri(octadecyloxy)benzyl alcohol.

In some embodiments, the lipophilic molecular anchor group is derived from 2,4-di(docosyloxy)benzyl alcohol.

In some embodiments, the lipophilic molecular anchor group is derived from 4-methoxy-2-[3′,4′,5′-tri(octadecyloxy)benzyloxy]benzyl alcohol.

In some embodiments, the lipophilic molecular anchor group is derived from 4-methoxy-2-[3′,4′,5′-tri(octadecyloxy)cyclohexylmethyloxy]benzyl alcohol.

In some embodiments, the lipophilic molecular anchor group is derived from 2-methoxy-4-[3′,4′,5′-tri(octadecyloxy)cyclohexylmethyloxy]benzyl alcohol.

In some embodiments, the lipophilic molecular anchor group is derived from 4-[3′,4′,5′-tri(octadecyloxy)cyclohexylmethyloxy]benzyl alcohol.

In some embodiments, the lipophilic molecular anchor group is derived from 3,5-dimethoxy-4-[3′,4′,5′-tri(octadecyloxy)cyclohexylmethyloxy]benzyl alcohol.

In some embodiments, the lipophilic molecular anchor group is derived from 2,4-di(dodecyloxy)benzyl alcohol.

In some embodiments, the lipophilic molecular anchor group is derived from 3,4,5-tri(octadecyloxy)benzylamine.

In some embodiments, the lipophilic molecular anchor group is derived from 2,4-bisoctadecyloxyphenylmethanol.

In some embodiments, the lipophilic molecular anchor group is derived from 2,4-bis-(2-decyl-tetradecyloxy)-phenylmethanol.

In some embodiments, the lipophilic molecular anchor group is derived from 2,3,4-trioctadecanoxybenzhydrol.

In some embodiments, the lipophilic molecular anchor group is derived from [phenyl(2,3,4-trioctadecanoxyphenyl)methyl]amine.

In some embodiments, the lipophilic molecular anchor group is derived from 4,4′-didocosoxybenzhydrol.

In some embodiments, the lipophilic molecular anchor group is derived from di(4-docosoxyphenyl)methylamine.

In some embodiments, the lipophilic molecular anchor group is derived from 4,4-di(12-docosoxydodecyloxy)benzhydrol.

In some embodiments, the lipophilic molecular anchor group is derived from amino-bis[4-(12-docosoxydodecyloxy)phenyl]methane.

In some embodiments, the lipophilic molecular anchor group is derived from N-benzyl-[bis(4-docosyloxyphenyl)]methylamine.

In some embodiments, the lipophilic molecular anchor group is derived from (4-methoxy-phenyl)-[4-(3,4,5-tris-octadecyloxy-cyclohexylmethoxy)-phenyl]-methanol.

In some embodiments, the lipophilic molecular anchor group is derived from {(4-methoxy-phenyl)-[4-(3,4,5-tris-octadecyloxy-cyclohexylmethoxy)-phenyl]-methyl}-amine.

In some embodiments, the lipophilic molecular anchor group is derived from [bis-(4-docosoxy-phenyl)-methyl]-amine.

In some embodiments, the lipophilic molecular anchor group is derived from 4-(12′-docosyloxy-1′-dodecyloxy)benzyl alcohol.

In some embodiments, the lipophilic molecular anchor group is derived from 4-(12′-docosyloxy-1′-dodecyloxy)-2-methoxybenzyl alcohol.

In some embodiments, the lipophilic molecular anchor group is derived from 4-(12′-docosyloxy-1′-dodecyloxy)-2-methoxybenzylamine.

In some embodiments, the lipophilic molecular anchor group is derived from 2-(12′-docosyloxy-1′-dodecyloxy)-4-methoxybenzyl alcohol.

In some embodiments, the lipophilic molecular anchor group is derived from 2-(12′-docosyloxy-1′-dodecyloxy)-4-methoxybenzylamine.

In some embodiments, the lipophilic molecular anchor group is derived from 2-docosyloxy-4-methoxybenzyl alcohol.

In some embodiments, the lipophilic molecular anchor group is derived from 4-methoxy-2-[3′,4′,5′-tris(octadecyloxy)benzyloxy)benzyl alcohol.

In some embodiments, the lipophilic molecular anchor group is derived from 2-[3′,5′-di(docosyloxy)benzyloxy]-4-methoxybenzyl alcohol.

In some embodiments, the lipophilic molecular anchor group is derived from 2-methoxy-4-[2′,2′,2′-tris(octadecyloxymethyl)ethoxy)benzyl alcohol.

In some embodiments, the lipophilic molecular anchor group is derived from 2-methoxy-4-[2′,2′,2′-tris(octadecyloxymethyl)ethoxy]benzylamine.

In some embodiments, the lipophilic molecular anchor group is derived from 4-methoxy-2-[3′,4′,5′-tris(octadecyloxy)cyclohexylmethyloxy]benzyl alcohol.

In some embodiments, the lipophilic molecular anchor group is derived from 2-methoxy-4-[3′,4′,5′-tris(octadecyloxy)cyclohexylmethyloxy]benzyl alcohol.

In some embodiments, the lipophilic molecular anchor group is derived from 4-[3′,4′,5′-tris(octadecyloxy)cyclohexylmethyloxy]benzyl alcohol.

In some embodiments, the lipophilic molecular anchor group is derived from 3,5-dimethoxy-4-[3′,4′,5′-tris(octadecyloxy)cyclohexylmethyloxy]benzyl alcohol.

In some embodiments, the lipophilic molecular anchor group is derived from N-(4-hydroxymethyl-3-methoxyphenyl) 3,4,5-tris(octadecyloxy)cyclohexylcarboxamide.

In some embodiments, the lipophilic molecular anchor group is derived from N-(5-hydroxymethyl-2-methoxyphenyl) 3,4,5-tris(octadecyloxy)cyclohexylcarboxamide.

In some embodiments, the lipophilic molecular anchor group is derived from N-(4-hydroxymethylphenyl) 3,4,5-tris(octadecyloxy)cyclohexylcarboxamide.

In some embodiments, the lipophilic molecular anchor group is derived from 1,22-bis[12-(4-hydroxymethyl-3-methoxyphenoxy)dodecyloxy]docosane.

In some embodiments, the lipophilic molecular anchor group is derived from 1,22-bis[12-(2-hydroxymethyl-5-methoxyphenoxy)dodecyloxy]docosane.

### Benzyl Alcohol Anchor Groups

A benzyl alcohol anchor group is a lipophilic molecular anchor group that is derived from a substituted benzyl alcohol. Example benzyl alcohol anchor groups useful in methods of the present disclosure include, but are not limited to, lipophilic molecular anchor groups derived from a compound selected from:
3,4,5-tri(octadecyloxy)benzyl alcohol;
2,4-di(docosyloxy)benzyl alcohol;
4-methoxy-2-[3′,4′,5′-tri(octadecyloxy)benzyloxy]benzyl alcohol;
4-methoxy-2-[3′,4′,5′-tri(octadecyloxy)cyclohexylmethyloxy]benzyl alcohol;
2-methoxy-4-[3′,4′,5′-tri(octadecyloxy)cyclohexylmethyloxy]benzyl alcohol;
4-[3′,4′,5′-tri(octadecyloxy)cyclohexylmethyloxy]benzyl alcohol;
3,5-dimethoxy-4-[3′,4′,5′-tri(octadecyloxy)cyclohexylmethyloxy]benzyl alcohol;
2,4-di(dodecyloxy)benzyl alcohol;
2,4-bisoctadecyloxyphenylmethanol;
2,4-bis-(2-decyl-tetradecyloxy)-phenylmethanol;
4-(12′-docosyloxy-1′-dodecyloxy)benzyl alcohol;
4-(12′-docosyloxy-1′-dodecyloxy)-2-methoxybenzyl alcohol;
2-(12′-docosyloxy-1′-dodecyloxy)-4-methoxybenzyl alcohol;
2-docosyloxy-4-methoxybenzyl alcohol;
4-methoxy-2-[3′,4′,5′-tris(octadecyloxy)benzyloxy)benzyl alcohol;
2-[3′,5′-di(docosyloxy)benzyloxy]-4-methoxybenzyl alcohol;
2-methoxy-4-[2′,2′,2′-tris(octadecyloxymethyl)ethoxy)benzyl alcohol;
4-methoxy-2-[3′,4′,5′-tris(octadecyloxy)cyclohexylmethyloxy]benzyl alcohol;
2-methoxy-4-[3′,4′,5′-tris(octadecyloxy)cyclohexylmethyloxy]benzyl alcohol;
4-[3′,4′,5′-tris(octadecyloxy)cyclohexylmethyloxy]benzyl alcohol;
3,5-dimethoxy-4-[3′,4′,5′-tris(octadecyloxy)cyclohexylmethyloxy]benzyl alcohol;
N-(4-hydroxymethyl-3-methoxyphenyl) 3,4,5-tris(octadecyloxy)cyclohexylcarboxamide;
N-(5-hydroxymethyl-2-methoxyphenyl) 3,4,5-tris(octadecyloxy)cyclohexylcarboxamide;
N-(4-hydroxymethylphenyl) 3,4,5-tris(octadecyloxy)cyclohexylcarboxamide;
1,22-bis[12-(4-hydroxymethyl-3-methoxyphenoxy)dodecyloxy]docosane; and
1,22-bis[12-(2-hydroxymethyl-5-methoxyphenoxy)dodecyloxy]docosane.

In some embodiments of the present disclosure, the C-terminal carboxy group of a C-protected amino acid or C protected peptide is protected by a benzyl alcohol anchor group.

In some embodiments, the benzyl alcohol anchor group is derived from 3,4,5-tri(octadecyloxy)benzyl alcohol.

In some embodiments, the benzyl alcohol anchor group is

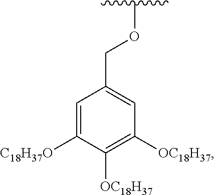

which may be referred to as the “C18 tag” or “C18 anchor group” herein.

A benzyl alcohol anchor group, such as, e.g., 3,4,5-tri(octadecyloxy)benzyl alcohol, can be coupled to an amino acid or a peptide via an esterification reaction to produce a C-protected amino acid or a C-protected peptide.

In some embodiments, the esterification reaction is performed in a solvent selected from 2-MeTHF, THF, toluene, and combinations of any of the foregoing. In some embodiments, the solvent is 2-MeTHF. In some embodiments, the solvent is THF. In some embodiments, the solvent is toluene.

In some embodiments, the esterification reaction is performed using N,N-diisopropylethylamine (DIPEA), 4-(N,N-dimethylamino)pyridine (DMAP), and propanephosphonic acid anhydride (T3P). In some embodiments, the esterification reaction is performed using a 1:2 molar ratio of DIPEA to DMAP.

In some embodiments, the esterification reaction is performed using DMAP and DIC. In some embodiments, the esterification reaction is performed using a 1:2 molar ratio of DMAP to DIC. In some embodiments, the esterification reaction is performed using a 1:4 molar ratio of DMAP to DIC.

In some embodiments, the esterification reaction is performed using DMAP and EDC. In some embodiments, the esterification reaction is performed using a 1:2 molar ratio of DMAP to EDC. In some embodiments, the esterification reaction is performed using a 1:4 molar ratio of DMAP to EDC.

As described herein, benzyl alcohol anchor groups can be selectively removed from a C-protected peptide or C-protected amino acid using lithium hydroxide. Lithium hydroxide can be used to hydrolyze the ester linkage between the substituted benzyl alcohol and the amino acid or peptide, yielding an amino acid or peptide in which the C-terminal carboxy group is not protected.

### Protecting Groups for Side Chain Functional Groups

The side chains of natural and unnatural amino acids include a variety of functional groups that can result in side chain reactivity during peptide synthesis. To minimize side chain reactivity, side chain protecting groups can be used. Side chain protecting groups are well-known in the art and designed to withstand multiple peptide elongation cycles. Specifically, side chain protecting groups used in the methods described herein are stable under 9-fluorenylmethyloxycarbonyl (Fmoc) deprotection conditions, such as exposure to a mild base.

For example, in some embodiments, side chain protecting groups that can be used in methods of the present disclosure include trityl (Trt), tert-butoxycarbonyl (Boc), tert-butyl (tBu), and benzothiophenesulfone-2-methyloxycarbonyl (Bsmoc).

Illustratively, amino protecting groups that can be used in methods of the present disclosure include, but are not limited to, urethane-type protecting groups (e.g., methoxycarbonyl, ethoxycarbonyl, tert-butoxycarbonyl (Boc), benzyloxycarbonyl (Cbz)), acyl-type protecting groups (e.g., formyl, acetyl, trifluoroacetyl), and sulfonyl-type protecting groups (e.g., p-toluenesulfonyl (Ts), p-tolylmethanesulfonyl, 4-methoxy-2,3,6-trimethylbenzenesulfonyl).

Hydroxy protecting groups that can be used in the disclosed methods include, but are not limited to, alkyl-type protecting groups (e.g., ethyl, ethyl, tert-butyl (tBu)), alkoxylalkyl-type protecting groups (e.g., methoxymethyl (MOM), 2-tetrahydropyranyl (THP), ethoxyethyl (EE)), acyl-type protecting groups (e.g., acetyl, pivaloyl, benzoyl), alkylsilyl-type protecting groups (e.g., trimethylsilyl (TMS), triethylsilyl (TES), tert-butyldimethylsilyl (TBS, TBDMS), triisopropyl silyl (TIPS), tert-butyldiphenylsilyl (TBDPS)), allyl protecting groups, and o-nitrobenzyl.

Arginine protecting groups that can be used in the disclosed methods include, but are not limited to, p-toluenesulfonyl and 2,2,4,6,7-pentamethyldihydrobenzofuran-5-sulfonyl (Pbf).

Protecting groups for asparagine, glutamine, and histidine that can be used in the disclosed methods include, but are not limited to, trityl and benzyloxymethyl.

Cysteine protecting groups that can be used in the disclosed methods include, but are not limited to, trityl, p-methylbenzyl, and acetamidomethyl.

Tryptophan protecting groups that can be used in the disclosed methods include, but are not limited to, Boc and formyl.

### Condensation Reactions

The coupling reagents that can be used to condense an N-terminal amino group of a C-protected amino acid or a C-protected peptide with a C-terminal carboxy group of an N-Fmoc amino acid or an N-Fmoc peptide in synthetic methods of the present disclosure are not particularly limited. Illustratively, N,N′-dicyclohexylcarbodiimide (DCC), N,N′-diisopropylcarbodiimide (DIC), N-(3-dimethylaminopropyl)-N′-ethylcarbodiimide (EDC), (benzotriazol-1-yloxy)tripyrrolidinophosphonium hexafluorophosphate (PyBOP), [ethyl cyano(hydroxyimino)acetato-O²]tri-1-pyrrolidinylphosphonium hexafluorophosphate (PyOxim), bromo-tris-pyrrolidino-phosphonium hexafluorophosphate (PyBroP), 7-azabenzotriazol-1-yloxy)tripyrrolidinophosphonium hexafluorophosphate (PyAOP), O-(benzotriazol-1-yl)-N,N,N′,N′-tetramethyluronium tetrafluoroborate (TBTU), O-(1H-6-chlorobenzotriazole-1-yl)-1,1,3,3-tetramethyluronium hexafluorophosphate (HCTU), 2-(1H-benzotriazole-1-yl)-1,1,3,3-tetramethyluronium hexafluorophosphate (HBTU), 1-hydroxybenzotriazole (HOBt), ethyl 1-hydroxy-1H-1,2,3-triazole-4-carboxylate (HOCt), 1-hydroxy-7-azabenzotriazole (HOAt), 4-dimethylaminopyridine (DMAP), 1-cyano-2-ethoxy-2-oxoethylidenaminooxy)dimethylamino-morpholino-carbenium hexafluorophosphate (COMU), O-[(ethoxycarbonyl)cyanomethylenamino]-N,N,N′,N′-tetra methyluronium tetrafluoroborate (TOTU), and combinations of the foregoing can be used as coupling reagents in condensation reactions described herein.

In some embodiments, the coupling reagent(s) used to condense an N-terminal amino group of a C-protected amino acid or a C-protected peptide with a C-terminal carboxy group of an N-Fmoc amino acid or an N-Fmoc peptide include a condensing agent, such as, e.g., DCC, DIC, EDC, PyBOP, TBTU, HCTU, or HBTU, and an activating agent, such as, e.g., HOBt, HOCt, HOAt, HBTU, HCTU, or DMAP. In some embodiments, the coupling reagents comprise EDC and HOBt. In some embodiments, the coupling reagents comprise EDC and DMAP. In some embodiments, the coupling reagents comprise DIC and HOBt. In some embodiments, the coupling reagents comprise DIC and DMAP.

In each condensation reaction of methods of the present disclosure, the amount of N-Fmoc amino acid or an N-Fmoc peptide used is not particularly limited. In some embodiments, the amount of N-Fmoc amino acid or N-Fmoc peptide used is sufficient to react with all present C-protected amino acid or C-protected peptide to avoid potential deletion of an amino acid in the synthesized peptide. For example, in some embodiments, the ratio (molar equivalents) of N-Fmoc amino acid/N-Fmoc peptide to C-protected amino acid/C-protected peptide is in the range of greater than 1 to 8, such as, e.g., greater than 1 to 4, greater than 1 to 2, or greater than 1 to 1.5. In some embodiments, the ratio of N-Fmoc amino acid/N-Fmoc peptide to C-protected amino acid/C-protected peptide is at least 1.01, at least 1.03, at least 1.05, at least 1.1, at least 1.2, at least 1.3, at least 1.4, at least 1.5, at least 1.6, at least 1.7, at least 1.8, at least 1.9, at least 2, at least 2.5, at least 3, at least 3.5, at least 4, at least 4.5, at least 5, at least 5.5, at least 6, at least 6.5, at least 7, or at least 7.5. In some embodiments, the ratio of N-Fmoc amino acid/N-Fmoc peptide to C-protected amino acid/C-protected peptide is 1.01, 1.03, 1.05, 1.1, 1.2, 1.3, 1.4, 1.5, 1.6, 1.7, 1.8, 1.9, 2, 2.25, 2.5, 2.75, 3, 3.25, 3.5, 3.75, 4, 4.25, 4.5, 4.75, 5, 5.25, 5.5, 5.75, 6, 6.25, 6.5, 6.75, 7, 7.25, 7.5, 7.75, or 8.

In certain condensation reactions described herein, the ratio (molar equivalents) of condensing agent to N-Fmoc amino acid/N-Fmoc peptide can be in the range of 0.7 to 1.5, such as, e.g., 0.85 to 1. In addition, the ratio (molar equivalents) of activating agent to N-Fmoc amino acid/N-Fmoc peptide can be in the range of 0 to 4, such as, e.g., 0.1 to 1.5 or 1, in condensation reactions described herein.

In certain methods of the present disclosure, each condensation reaction can be independently performed at a temperature in the range of −10° C. to 50° C. (such as, e.g., −10° C. to 30° C., 0° C. to 30° C., 0° C. to 20° C., 0° C. to 10° C., 20° C. to 40° C., 20° C. to 30° C.). In some embodiments, condensing an N-terminal amino group of a C-protected amino acid or a C-protected peptide with a C-terminal carboxy group of an N-Fmoc amino acid or an N-Fmoc peptide occurs at a temperature in the range of 0° C. to 40° C. In some embodiments, the condensing occurs at a temperature in the range of 10° C. to 40° C. In some embodiments, the condensing occurs at a temperature in the range of 20° C. to 40° C. In some embodiments, the condensing occurs at a temperature in the range of 20° C. to 30° C. In some embodiments, the condensing occurs at a temperature of 25° C.

Each condensation reaction can proceed until a desired level of conversion is achieved. For example, in some embodiments, a condensation reaction can proceed for a time in the range of 1 hour to 72 hours, such as, e.g., 1 hour to 30 hours, 2 hours to 24 hours, 12 hours to 24 hours, or overnight.

### Washing and Separation of N-Fmoc C-Protected Peptides

Following a condensation reaction, a reaction mixture comprising an N-Fmoc C-protected peptide can be washed with an aqueous solution, such as a brine solution, one or more times to separate an organic layer comprising the N-Fmoc C-protected peptide from an aqueous layer comprising unreacted coupling reagents and excess N-Fmoc amino acids or N-Fmoc peptides. In some embodiments, the reaction mixture is washed once. In some embodiments, the reaction mixture is washed once with a brine solution.

Optionally, an amine scavenger can be included in the aqueous wash solution to capture unreacted amino acid esters. Non-limiting examples of water-soluble amines that can be used as amine scavengers include 1-methylpiperazine, 4-aminopiperidine, diethylenetriamine, triaminoethylamine, 1-ethylpiperazine, N,N-dimethylethylenediamine, ethylenediamine, and piperazine. In some embodiments, 1-10 (e.g., 1-6, 1-4) molar equivalents of amine scavenger relative to the amount of unreacted amino acid esters theoretically remaining after condensation can be added to the aqueous solution.

### Fmoc Deprotection of N-Fmoc C-Protected Peptides

The N-Fmoc C-protected peptide can be Fmoc deprotected in the separated organic layer (i.e., in a solvent system comprising 2-MeTHF) to obtain a reaction mixture comprising a C-protected peptide. In some embodiments of the present disclosure, the N-terminal Fmoc group can be removed with a non-nucleophilic organic base in the presence of an acidic thiol.

In some embodiments, the non-nucleophilic organic base is selected from N,N-diisopropylethylamine, 1,8-diazabicyclo[5.4.0]undec-7-ene (DBU), 1,4-diazabicyclo[2.2.2]octane (DABCO), and 1,5-diazabicyclo[4.3.0]non-5-ene (DBN). In some embodiments, the non-nucleophilic organic base is N,N-diisopropylethylamine. In some embodiments, the non-nucleophilic organic base is 1,8-diazabicyclo[5.4.0]undec-7-ene (DBU). In some embodiments, the non-nucleophilic organic base is 1,4-diazabicyclo[2.2.2]octane (DABCO). In some embodiments, the non-nucleophilic organic base is 1,5-diazabicyclo[4.3.0]non-5-ene (DBN).

In some embodiments, the acidic thiol is selected from cysteine, thiomalic acid, and mercaptopropionic acid. In some embodiments, the acidic thiol is cysteine. In some embodiments, the acidic thiol is thiomalic acid. In some embodiments, the acidic thiol is mercaptopropionic acid.

In some embodiments, the non-nucleophilic organic base is DBU and the acidic thiol is cysteine. In some embodiments, the non-nucleophilic organic base is DBU and the acidic thiol is thiomalic acid. In some embodiments, the non-nucleophilic organic base is DBU and the acidic thiol is mercaptopropionic acid.

In some embodiments, 0.8-100 mol (e.g., 1-10 mol, 1-5 mol, 1-3 mol) of non-nucleophilic organic base is used per 1 mol of N-Fmoc C-protected peptide for Fmoc deprotection. In some embodiments, 1-10 mol of non-nucleophilic organic base is used per 1 mol of N-Fmoc C-protected peptide for Fmoc deprotection. In some embodiments, 1 mol of non-nucleophilic organic base is used per 1 mol of N-Fmoc C-protected peptide for Fmoc deprotection. In some embodiments, 2 mol of non-nucleophilic organic base is used per 1 mol of N-Fmoc C-protected peptide for Fmoc deprotection. In some embodiments, 3 mol of non-nucleophilic organic base is used per 1 mol of N-Fmoc C-protected peptide for Fmoc deprotection. In some embodiments, 4 mol of non-nucleophilic organic base is used per 1 mol of N-Fmoc C-protected peptide for Fmoc deprotection. In some embodiments, 5 mol of non-nucleophilic organic base is used per 1 mol of N-Fmoc C-protected peptide for Fmoc deprotection. In some embodiments, 6 mol of non-nucleophilic organic base is used per 1 mol of N-Fmoc C-protected peptide for Fmoc deprotection. In some embodiments, 7 mol of non-nucleophilic organic base is used per 1 mol of N-Fmoc C-protected peptide for Fmoc deprotection. In some embodiments, 8 mol of non-nucleophilic organic base is used per 1 mol of N-Fmoc C-protected peptide for Fmoc deprotection. In some embodiments, 9 mol of non-nucleophilic organic base is used per 1 mol of N-Fmoc C-protected peptide for Fmoc deprotection. In some embodiments, 10 mol of non-nucleophilic organic base is used per 1 mol of N-Fmoc C-protected peptide for Fmoc deprotection.

In some embodiments, the ratio (molar equivalents) of non-nucleophilic organic base to acidic thiol used for Fmoc deprotection is 3:1. In some embodiments, the ratio of non-nucleophilic organic base to acidic thiol used for Fmoc deprotection is 2.5:1. In some embodiments, the ratio of non-nucleophilic organic base to acidic thiol used for Fmoc deprotection is 2:1.

In certain methods of the present disclosure, each Fmoc deprotection step can be independently performed at a temperature in the range of −10° C. to 50° C. (such as, e.g., −10° C. to 30° C., 0° C. to 30° C., 0° C. to 20° C.). In some embodiments, the Fmoc deprotection occurs at a temperature in the range of 0° C. to 40° C. In some embodiments, the Fmoc deprotection occurs at a temperature in the range of 0° C. to 30° C. In some embodiments, the Fmoc deprotection occurs at a temperature in the range of 10° C. to 20° C. In some embodiments, the Fmoc deprotection occurs at a temperature of 15° C. In some embodiments, the Fmoc deprotection occurs at a temperature of 25° C. In some embodiments, the Fmoc deprotection occurs at ambient temperature.

Additionally, in some embodiments of the present disclosure, each Fmoc deprotection step can independently proceed for a time in the range of 5 minutes to 72 hours, e.g., 10 minutes to 30 minutes, 1 hour to 30 hours, or 2 hours. In some embodiments, each Fmoc deprotection step can independently proceed for a time in the range of 10 minutes to 30 minutes.

### Optional Acid Neutralization

Use of an excess amount of non-nucleophilic organic base for Fmoc deprotection may adversely affect subsequent steps. Accordingly, following Fmoc deprotection with a non-nucleophilic organic base, the reaction mixture comprising the C-protected peptide can optionally be neutralized using an acidic solution prior to washing and separation. In some embodiments, at least 0.5 mol (e.g., at least 0.9, 0.5-10, 0.5-5) acid per 1 mol of non-nucleophilic organic base is used for acid neutralization. Example acids that can be used for neutralization include, but are not limited to, methanesulfonic acid, trifluoromethanesulfonic acid, benzenesulfonic acid, p toluenesulfonic anhydride, sulfuric acid, and hydrogen chloride.

In some embodiments, the acidic solution comprises an acid selected from methanesulfonic acid, trifluoromethanesulfonic acid, benzenesulfonic acid, p toluenesulfonic anhydride, sulfuric acid, hydrogen chloride, and combinations of any of the foregoing. In some embodiments, the acidic solution comprises methanesulfonic acid. In some embodiments, the acidic solution comprises trifluoromethanesulfonic acid. In some embodiments, the acidic solution comprises benzenesulfonic acid. In some embodiments, the acidic solution comprises p-toluenesulfonic anhydride. In some embodiments, the acidic solution comprises sulfuric acid. In some embodiments, the acidic solution comprises hydrogen chloride.

In some embodiments, the pH of the acidic solution is in the range of 1 to 5. In some embodiments, the pH of the acidic solution is in the range of 1 to 4. In some embodiments, the pH of the acidic solution is in the range of 1 to 3. In some embodiments, the pH of the acidic solution is 1, 1.5, 2, 2.5, 3, 3.5, 4, 4.5, or 5. In some embodiments, the pH of the acidic solution is 1. In some embodiments, the pH of the acidic solution is 1.5. In some embodiments, the pH of the acidic solution is 2. In some embodiments, the pH of the acidic solution is 2.5. In some embodiments, the pH of the acidic solution is 3. In some embodiments, the pH of the acidic solution is 3.5. In some embodiments, the pH of the acidic solution is 4. In some embodiments, the pH of the acidic solution is 4.5. In some embodiments, the pH of the acidic solution is 5.

In other embodiments of the present disclosure, the reaction mixture obtained from the Fmoc deprotection step is washed with an aqueous solution and phase separated without an intervening acid neutralization.

### Washing and Separation of C-Protected Peptides

After Fmoc deprotection and optional acid neutralization, the C-protected peptide can be isolated by washing with one or more aqueous solutions. If more than one aqueous wash solution is used, each aqueous wash solution may have the same composition or a different composition.

In some embodiments, the reaction mixture comprising the C-protected peptide is washed with an aqueous solution comprising sodium carbonate. In some embodiments, the reaction mixture comprising the C-protected peptide is washed with an aqueous solution comprising sodium carbonate at a concentration of 10% (v/v).

In some embodiments, the reaction mixture comprising the C-protected peptide is washed with one aqueous solution comprising sodium carbonate. In some embodiments, the reaction mixture comprising the C-protected peptide is washed with one aqueous solution comprising sodium carbonate at a concentration of 10% (v/v).

In some embodiments, the reaction mixture comprising the C-protected peptide is washed with an aqueous solution comprising sodium carbonate and dimethylformamide (DMF). In some embodiments, the reaction mixture comprising the C-protected peptide is washed with an aqueous solution comprising sodium carbonate at a concentration of 10% (v/v) and DMF. In some embodiments, the aqueous solution comprises sodium carbonate and DMF at a 5:1 ratio.

In some embodiments, the reaction mixture comprising the C-protected peptide is washed with one aqueous solution comprising sodium carbonate and DMF. In some embodiments, the reaction mixture comprising the C-protected peptide is washed with one aqueous solution comprising sodium carbonate at a concentration of 10% (v/v) and DMF. In some embodiments, the aqueous solution comprises sodium carbonate and DMF at a 5:1 ratio.

In some embodiments, the reaction mixture comprising the C-protected peptide is washed with an aqueous solution comprising sodium carbonate and optionally DMF and with one or more saturated sodium chloride solutions. In some embodiments, the reaction mixture comprising the C-protected peptide is washed with an aqueous solution comprising sodium carbonate and with one or more saturated sodium chloride solutions. In some embodiments, the reaction mixture comprising the C-protected peptide is washed with an aqueous solution comprising sodium carbonate at a concentration of 10% (v/v) and with one or more saturated sodium chloride solutions. In some embodiments, the reaction mixture comprising the C-protected peptide is washed with an aqueous solution comprising sodium carbonate and DMF and with one or more saturated sodium chloride solutions. In some embodiments, the reaction mixture comprising the C-protected peptide is washed with an aqueous solution comprising sodium carbonate at a concentration of 10% (v/v) and DMF and with one or more saturated sodium chloride solutions.

In some embodiments, the reaction mixture comprising the C-protected peptide is washed with one aqueous solution comprising sodium carbonate and optionally DMF and with one or more saturated sodium chloride solutions. In some embodiments, the reaction mixture comprising the C-protected peptide is washed with one aqueous solution comprising sodium carbonate and with one or more saturated sodium chloride solutions. In some embodiments, the reaction mixture comprising the C-protected peptide is washed with one aqueous solution comprising sodium carbonate at a concentration of 10% (v/v) and with one or more saturated sodium chloride solutions. In some embodiments, the reaction mixture comprising the C-protected peptide is washed with one aqueous solution comprising sodium carbonate and DMF and with one or more saturated sodium chloride solutions. In some embodiments, the reaction mixture comprising the C-protected peptide is washed with one aqueous solution comprising sodium carbonate at a concentration of 10% (v/v) and DMF and with one or more saturated sodium chloride solutions.

### Iterative Liquid Phase Peptide Synthesis Processes

In certain methods of the present disclosure, a cycle of peptide elongation comprising condensation, washing, Fmoc deprotection, and washing can be performed in one pot. Moreover, in some methods of the present disclosure, multiple cycles of peptide elongation can be performed by repeating the general steps of condensation, washing, Fmoc deprotection, and washing, where the specific conditions for each step in each cycle (e.g., reaction temperature, reaction time, choice and ratio of reagents) can be independently selected. These multiple cycles can also be performed in one pot without, for example, requiring any material to be isolated by precipitation.

In some embodiments, one or more (e.g., two or more, three or more, four or more, five or more, six or more, seven or more, eight or more, nine or more, ten or more) cycles of peptide elongation are performed. In some embodiments, one or more cycles of peptide elongation are performed. In some embodiments, two or more cycles of peptide elongation are performed. In some embodiments, three or more cycles of peptide elongation are performed. In some embodiments, four or more cycles of peptide elongation are performed. In some embodiments, five or more cycles of peptide elongation are performed. In some embodiments, six or more cycles of peptide elongation are performed. In some embodiments, seven or more cycles of peptide elongation are performed. In some embodiments, eight or more cycles of peptide elongation are performed. In some embodiments, nine or more cycles of peptide elongation are performed. In some embodiments, ten or more cycles of peptide elongation are performed.

In some embodiments, one or more (e.g., two or more, three or more, four or more, five or more, six or more, seven or more, eight or more, nine or more, ten or more) cycles of peptide elongation are performed in one pot. In some embodiments, one or more cycles of peptide elongation are performed in one pot. In some embodiments, two or more cycles of peptide elongation are performed in one pot. In some embodiments, three or more cycles of peptide elongation are performed in one pot. In some embodiments, four or more cycles of peptide elongation are performed in one pot. In some embodiments, five or more cycles of peptide elongation are performed in one pot. In some embodiments, six or more cycles of peptide elongation are performed in one pot. In some embodiments, seven or more cycles of peptide elongation are performed in one pot. In some embodiments, eight or more cycles of peptide elongation are performed in one pot. In some embodiments, nine or more cycles of peptide elongation are performed in one pot. In some embodiments, ten or more cycles of peptide elongation are performed in one pot.

In some embodiments, one, two, three, four, five, six, seven, eight, nine, or ten cycles of peptide elongation are performed. In some embodiments, one cycle of peptide elongation is performed. In some embodiments, two cycles of peptide elongation are performed. In some embodiments, three cycles of peptide elongation are performed. In some embodiments, four cycles of peptide elongation are performed. In some embodiments, five cycles of peptide elongation are performed. In some embodiments, six cycles of peptide elongation are performed. In some embodiments, seven cycles of peptide elongation are performed. In some embodiments, eight cycles of peptide elongation are performed. In some embodiments, nine cycles of peptide elongation are performed. In some embodiments, ten cycles of peptide elongation are performed.

In some embodiments, one, two, three, four, five, six, seven, eight, nine, or ten cycles of peptide elongation are performed in one pot. In some embodiments, one cycle of peptide elongation is performed in one pot. In some embodiments, two cycles of peptide elongation are performed in one pot. In some embodiments, three cycles of peptide elongation are performed in one pot. In some embodiments, four cycles of peptide elongation are performed in one pot. In some embodiments, five cycles of peptide elongation are performed in one pot. In some embodiments, six cycles of peptide elongation are performed in one pot. In some embodiments, seven cycles of peptide elongation are performed in one pot. In some embodiments, eight cycles of peptide elongation are performed in one pot. In some embodiments, nine cycles of peptide elongation are performed in one pot. In some embodiments, ten cycles of peptide elongation are performed in one pot.

### Isolation by Precipitation

After one or more cycles of peptide elongation have been performed, a C-protected peptide can be isolated following Fmoc deprotection. Specifically, a polar solvent can be added to an organic layer comprising the C-protected peptide to precipitate the C-protected peptide, which can then be isolated by solid-liquid separation.

Non-limiting examples of polar solvents that can be used to precipitate a C-protected peptide include methanol (MeOH), ethanol (EtOH), isopropyl alcohol (IPA), acetic acid (AcOH), acetonitrile (ACN), acetone, isopropyl acetate (iPrOAc), N-methyl-2-pyrrolidone (NMP), dimethyl sulfoxide (DMSO), dimethylformamide (DMF), heptanes, dichloromethane (DCM), cyclopentyl methyl ether (CPME), and combinations of any of the foregoing. For example, in some embodiments, water, MeOH, EtOH, IPA, AcOH, ACN, acetone, NMP, DMSO, DMF, DCM, or combinations of any of the foregoing can be used to precipitate the C-protected peptide.

In some embodiments, the C-protected peptide is precipitated in a polar solvent system comprising water. In some embodiments, the C-protected peptide is precipitated in a polar solvent system comprising MeOH. In some embodiments, the C-protected peptide is precipitated in a polar solvent system comprising EtOH. In some embodiments, the C-protected peptide is precipitated in a polar solvent system comprising IPA. In some embodiments, the C-protected peptide is precipitated in a polar solvent system comprising AcOH. In some embodiments, the C-protected peptide is precipitated in a polar solvent system comprising ACN. In some embodiments, the C-protected peptide is precipitated in a polar solvent system comprising acetone. In some embodiments, the C-protected peptide is precipitated in a polar solvent system comprising NMP. In some embodiments, the C-protected peptide is precipitated in a polar solvent system comprising DMSO. In some embodiments, the C-protected peptide is precipitated in a polar solvent system comprising DMF. In some embodiments, the C-protected peptide is precipitated in a polar solvent system comprising DCM.

In some embodiments, the C-protected peptide is precipitated in a polar solvent system comprising water and MeOH. In some embodiments, the C-protected peptide is precipitated in a polar solvent system comprising water and EtOH. In some embodiments, the C-protected peptide is precipitated in a polar solvent system comprising water and IPA. In some embodiments, the C-protected peptide is precipitated in a polar solvent system comprising water and AcOH. In some embodiments, the C-protected peptide is precipitated in a polar solvent system comprising water and ACN. In some embodiments, the C-protected peptide is precipitated in a polar solvent system comprising water and acetone. In some embodiments, the C-protected peptide is precipitated in a polar solvent system comprising water and NMP. In some embodiments, the C-protected peptide is precipitated in a polar solvent system comprising water and DMSO. In some embodiments, the C-protected peptide is precipitated in a polar solvent system comprising water and DMF. In some embodiments, the C-protected peptide is precipitated in a polar solvent system comprising water and DCM.

In some embodiments, the C-protected peptide is precipitated in a polar solvent system comprising water and a solvent selected from MeOH, EtOH, IPA, AcOH, ACN, acetone, NMP, DMSO, DMF, and DCM, wherein the water is present at a concentration in the range of 10% (v/v) to 50% (v/v) and the solvent is present at a concentration of 50% (v/v) to 90% (v/v), and further wherein the total concentration of water and the solvent is 100% (v/v). In some embodiments, the solvent is present at a concentration in the range of 70% (v/v) to 90% (v/v). In some embodiments, the solvent is present at a concentration in the range of 75% (v/v) to 85% (v/v). In some embodiments, the solvent is present at a concentration of 80% (v/v).

In some embodiments, the C-protected peptide is precipitated in a 1:1 mixture of THF:water. In some embodiments, the C-protected peptide is precipitated in a 1:2 mixture of IPA:water. In some embodiments, the C-protected peptide is precipitated in a 2:1 mixture of ACN:water.

In some embodiments of the present disclosure, a precipitation accelerator can optionally be added to the polar solvent system to improve recovery (e.g., recovery rate, yield) of the C-protected peptide. Non-limiting examples of precipitation accelerators are described in U.S. Pat. No. 10,464,966.

In some embodiments, the precipitation of the C-protected peptide is performed at a temperature in the range of 10° C. to 40° C. (such as, e.g., 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C.). In some embodiments, the precipitation of the C-protected peptide is performed at ambient temperature.

In some embodiments, temperature is cycled for one or more cycles (such as, e.g., one, two, three, four, or five cycles) following anti-solvent addition, which may result in a more homogenous particle size distribution for the isolated C-protected peptide. In some embodiments, temperature is cycled between 40° C. and 25° C. for one or more cycles (such as, e.g., one, two, three, four, or five cycles) following anti-solvent addition.

### Lipophilic Molecular Anchor Group Removal

A lipophilic molecular anchor group can be removed from a C-protected peptide by a variety of means. For example, acid labile anchor groups can be removed by acid addition, which will also remove acid labile side chain protecting groups on the peptide and may serve, in some instances, as a global deprotection step for the C-protected peptide.

In some embodiments of the present disclosure, the lipophilic molecular anchor group is removed using an acid at a concentration in the range of 0.1% (w/v) to 5% (w/v). In some embodiments, the acid is selected from trifluoroacetic acid (TFA), hydrochloric acid, sulfuric acid, methanesulfonic acid, p-toluenesulfonic acid, and combinations of any of the foregoing. In some embodiments, the acid is TFA. In some embodiments, the acid is hydrochloric acid. In some embodiments, the acid is sulfuric acid. In some embodiments, the acid is methanesulfonic acid. In some embodiments, the acid is p-toluenesulfonic acid.

In alternative embodiments of the present disclosure, the lipophilic molecular anchor group can be removed from a C-protected peptide using a fluorine-substituted alcohol (e.g., trifluoroethanol (TFE), hexafluoroisopropanol (HFIP)) at a concentration in the range of 10% (w/v) to 100% (w/v). In some embodiments, the lipophilic molecular anchor group is removed using TFE. In some embodiments, the lipophilic molecular anchor group is removed using HFIP.

Additionally, in other alternative embodiments of the present disclosure, a benzyl alcohol anchor group, such as an anchor group derived from 3,4,5-tris(octadecyloxy)benzyl alcohol, can be selectively removed from a C-protected peptide by base-catalyzed ester hydrolysis.

In some embodiments, the base used for base-catalyzed ester hydrolysis is selected from hydroxides soluble in an organic solvent. In some embodiments, the base used for base-catalyzed ester hydrolysis is selected from sodium hydroxide, potassium hydroxide, lithium hydroxide, barium hydroxide, and tetrabutylammonium hydroxide. In some embodiments, the base used for base-catalyzed ester hydrolysis is sodium hydroxide. In some embodiments, the base used for base-catalyzed ester hydrolysis is potassium hydroxide. In some embodiments, the base used for base-catalyzed ester hydrolysis is lithium hydroxide. In some embodiments, the base used for base-catalyzed ester hydrolysis is barium hydroxide. In some embodiments, the base used for base-catalyzed ester hydrolysis is tetrabutylammonium hydroxide.

Illustratively, use of lithium hydroxide facilitates removal of the benzyl alcohol anchor group while maintaining side chain protecting groups on the peptide. To remove the benzyl alcohol anchor group, lithium hydroxide can be added to the C-protected peptide at a minimum of 1 molar equivalent (e.g., 1-4 eq., 1 eq., 2 eq., 3 eq., 4 eq.), e.g., in a solvent system comprising water and THF. In some embodiments, the solvent system comprises water and THF at a 1:4 volumetric ratio. In some embodiments, the selective anchor group removal reaction can be performed at a temperature in the range of 0° C. to 50° C. (e.g., 0° C. to 30° C.) and for a time in the range of 0.5 hour to 24 hours.

### Convergent Liquid Phase Peptide Synthesis Methods

Convergent liquid phase peptide synthesis methods described herein can be used to produce a desired peptide from fragments of the peptide, which can be coupled together via a convergent process. For example, following isolation of two or more C-protected peptides that are fragments of a desired peptide (“C-protected fragments”), the C-protected fragments can be coupled in a liquid environment to produce the desired peptide.

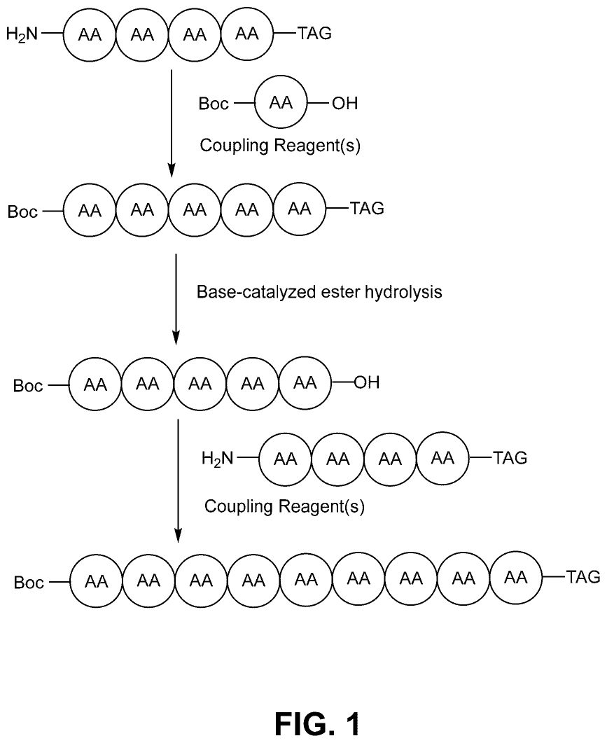

FIG. 1 depicts a non-limiting example process for the convergent assembly of two or more peptide fragments into a longer peptide. Specifically, two or more C-protected peptides can be prepared and isolated using, e.g., a liquid phase peptide synthesis process described herein. Following the synthesis and isolation of a C-protected peptide, a C-terminal carboxy group of an N-Boc amino acid can be condensed with an N-terminal amino group of a C-protected peptide to produce an N-Boc C-protected peptide. The lipophilic molecular anchor group protecting the C-terminus can then be selectively removed from the N-Boc C-protected peptide to produce an N-Boc peptide. Illustratively, when the lipophilic molecular anchor group is a benzyl alcohol anchor group (e.g., an anchor group derived from 3,4,5-tri(octadecyloxy)benzyl alcohol), the benzyl alcohol anchor group can be selectively removed by base-catalyzed ester hydrolysis as described herein. Next, a second C-protected peptide can be condensed with the N-Boc peptide to produce a second N-Boc C-protected peptide. The lipophilic molecular anchor group of the second N-Boc C-protected peptide can then be selectively removed and the process repeated iteratively to convergently assemble the desired peptide. After the final condensation reaction, the final N-Boc C-protected peptide can be globally deprotected by a variety of means, such as, e.g., by acid hydrolysis.

Some embodiments of the present disclosure relate to a method of producing a peptide comprising:
(i) condensing an N-terminal amino group of a first C-protected peptide with a C-terminal carboxy group of a first N-Boc amino acid or a first N-Boc peptide to obtain a first N-Boc C-protected peptide, wherein a C-terminal carboxy group of the first C-protected peptide is protected by a first benzyl alcohol anchor group;
(ii) removing the first benzyl alcohol anchor group from the first N-Boc C-protected peptide to obtain a second N-Boc peptide;
(iii) condensing a C-terminal carboxy group of the second N-Boc peptide with an N-terminal amino group of a second C-protected peptide to obtain a second N-Boc C-protected peptide, wherein a C-terminal carboxy group of the second C-protected peptide is protected by a second benzyl alcohol anchor group; and
(iv) removing the second benzyl alcohol anchor group from the second N-Boc C-protected peptide to obtain a third N-Boc peptide.

Example benzyl alcohol anchor groups useful in these methods include, but are not limited to, anchor groups derived from a compound selected from:
3,4,5-tri(octadecyloxy)benzyl alcohol;
2,4-di(docosyloxy)benzyl alcohol;
4-methoxy-2-[3′,4′,5′-tri(octadecyloxy)benzyloxy]benzyl alcohol;
4-methoxy-2-[3′,4′,5′-tri(octadecyloxy)cyclohexylmethyloxy]benzyl alcohol;
2-methoxy-4-[3′,4′,5′-tri(octadecyloxy)cyclohexylmethyloxy]benzyl alcohol;
4-[3′,4′,5′-tri(octadecyloxy)cyclohexylmethyloxy]benzyl alcohol;
3,5-dimethoxy-4-[3′,4′,5′-tri(octadecyloxy)cyclohexylmethyloxy]benzyl alcohol;
2,4-di(dodecyloxy)benzyl alcohol;
2,4-bisoctadecyloxyphenylmethanol;
2,4-bis-(2-decyl-tetradecyloxy)-phenylmethanol;
4-(12′-docosyloxy-1′-dodecyloxy)benzyl alcohol;
4-(12′-docosyloxy-1′-dodecyloxy)-2-methoxybenzyl alcohol;
2-(12′-docosyloxy-1′-dodecyloxy)-4-methoxybenzyl alcohol;
2-docosyloxy-4-methoxybenzyl alcohol;
4-methoxy-2-[3′,4′,5′-tris(octadecyloxy)benzyloxy)benzyl alcohol;
2-[3′,5′-di(docosyloxy)benzyloxy]-4-methoxybenzyl alcohol;
2-methoxy-4-[2′,2′,2′-tris(octadecyloxymethyl)ethoxy)benzyl alcohol;
4-methoxy-2-[3′,4′,5′-tris(octadecyloxy)cyclohexylmethyloxy]benzyl alcohol;
2-methoxy-4-[3′,4′,5′-tris(octadecyloxy)cyclohexylmethyloxy]benzyl alcohol;
4-[3′,4′,5′-tris(octadecyloxy)cyclohexylmethyloxy]benzyl alcohol;
3,5-dimethoxy-4-[3′,4′,5′-tris(octadecyloxy)cyclohexylmethyloxy]benzyl alcohol;
N-(4-hydroxymethyl-3-methoxyphenyl) 3,4,5-tris(octadecyloxy)cyclohexylcarboxamide;
N-(5-hydroxymethyl-2-methoxyphenyl) 3,4,5-tris(octadecyloxy)cyclohexylcarboxamide;
N-(4-hydroxymethylphenyl) 3,4,5-tris(octadecyloxy)cyclohexylcarboxamide;
1,22-bis[12-(4-hydroxymethyl-3-methoxyphenoxy)dodecyloxy]docosane; and
1,22-bis[12-(2-hydroxymethyl-5-methoxyphenoxy)dodecyloxy]docosane.

In some embodiments, the first benzyl alcohol anchor group is derived from 3,4,5-tri(octadecyloxy)benzyl alcohol. In some embodiments, the second benzyl alcohol anchor group is derived from 3,4,5-tri(octadecyloxy)benzyl alcohol. In some embodiments, the first benzyl alcohol anchor group and the second benzyl alcohol anchor group are each derived from 3,4,5-tri(octadecyloxy)benzyl alcohol.

In some embodiments, the first C-protected peptide is produced using a liquid phase peptide synthesis process. In some embodiments, the first C-protected peptide is produced using a liquid phase peptide synthesis process described herein.

In some embodiments, the second C-protected peptide is produced using a liquid phase peptide synthesis process. In some embodiments, the second C-protected peptide is produced using a liquid phase peptide synthesis process described herein.

In some embodiments, the first C-protected peptide and the second C-protected peptide are each produced using a liquid phase peptide synthesis process. In some embodiments, the first C-protected peptide and the second C-protected peptide are each produced using a liquid phase peptide synthesis process described herein.

In some embodiments, the (i) condensing comprises condensing the N-terminal amino group of the first C-protected peptide with the C-terminal carboxy group of the first N-Boc amino acid or the first N-Boc peptide in the presence of a first coupling reagent in a first solvent system to obtain a first reaction mixture comprising the first N-Boc C-protected peptide. Additionally, in some embodiments, the (iii) condensing comprises condensing the C-terminal carboxy group of the second N-Boc peptide with the N-terminal amino group of the second C-protected peptide in the presence of a second coupling reagent in a second solvent system to obtain a third reaction mixture comprising the second N-Boc C-protected peptide.

The coupling reagents that can be used to condense an N-terminal amino group of a C-protected peptide with a C-terminal carboxy group of an N-Boc amino acid or an N-Boc peptide in the (i) condensing and the (iii) condensing are not particularly limited. Moreover, the (iii) condensing can be performed under similar conditions to the (i) condensing (e.g., reagent(s), solvent(s), reaction time(s) and temperature(s)); however, the (i) and (iii) condensation reaction conditions can be independently selected.

Illustratively, N,N′-dicyclohexylcarbodiimide (DCC), N,N′-diisopropylcarbodiimide (DIC), N-(3-dimethylaminopropyl)-N′-ethylcarbodiimide (EDC), (benzotriazol-1-yloxy)tripyrrolidinophosphonium hexafluorophosphate (PyBOP), [ethyl cyano(hydroxyimino)acetato-O²]tri-1-pyrrolidinylphosphonium hexafluorophosphate (PyOxim), bromo-tris-pyrrolidino-phosphonium hexafluorophosphate (PyBroP), 7-azabenzotriazol-1-yloxy)tripyrrolidinophosphonium hexafluorophosphate (PyAOP), O-(benzotriazol-1-yl)-N,N,N′,N′-tetramethyluronium tetrafluoroborate (TBTU), O-(1H-6-chlorobenzotriazole-1-yl)-1,1,3,3-tetramethyluronium hexafluorophosphate (HCTU), 2-(1H-benzotriazole-1-yl)-1,1,3,3-tetramethyluronium hexafluorophosphate (HBTU), 1-hydroxybenzotriazole (HOBt), ethyl 1-hydroxy-1H-1,2,3-triazole-4-carboxylate (HOCt), 1-hydroxy-7-azabenzotriazole (HOAt), 4-dimethylaminopyridine (DMAP), 1-cyano-2-ethoxy-2-oxoethylidenaminooxy)dimethylamino-morpholino-carbenium hexafluorophosphate (COMU), O-[(ethoxycarbonyl)cyanomethylenamino]-N,N,N′,N′-tetra methyluronium tetrafluoroborate (TOTU), and combinations of the foregoing can be used as coupling reagents in the (i) and (iii) condensation reactions.

In some embodiments, the coupling reagent(s) used in the (i) condensing and/or the (iii) condensing include a condensing agent, such as, e.g., DCC, DIC, EDC, PyBOP, TBTU, HCTU, or HBTU, and an activating agent, such as, e.g., HOBt, HOCt, HOAt, HBTU, HCTU, or DMAP. In some embodiments, the coupling reagents comprise EDC and HOBt. In some embodiments, the coupling reagents comprise EDC and DMAP. In some embodiments, the coupling reagents comprise DIC and HOBt. In some embodiments, the coupling reagents comprise DIC and DMAP.

In some embodiments, in the (i) condensing, a molar excess (e.g., a 1.01-fold, a 1.1-fold, a 1.2 fold, a 1.3-fold, a 1.4-fold, a 1.5-fold, a 1.6-fold, a 1.7-fold, a 1.8-fold, a 1.9-fold, a 2-fold, a 3-fold, a 4-fold, a 5-fold, a 6-fold, a 7-fold, an 8-fold, a 9-fold, a 10-fold) of the first N-Boc amino acid or the first N-Boc peptide is used relative to the first C-protected peptide.

In some embodiments, in the (iii) condensing, a molar excess (e.g., a 1.01-fold, a 1.1-fold, a 1.2 fold, a 1.3-fold, a 1.4-fold, a 1.5-fold, a 1.6-fold, a 1.7-fold, a 1.8-fold, a 1.9-fold, a 2-fold, a 3-fold, a 4-fold, a 5-fold, a 6-fold, a 7-fold, an 8-fold, a 9-fold, a 10-fold) of the second N-Boc peptide is used relative to the second C-protected peptide.

In some embodiments, the (i) condensing and the (iii) condensing can be independently performed at a temperature in the range of −10° C. to 50° C. (such as, e.g., −10° C. to 30° C., 0° C. to 30° C., 0° C. to 20° C., 0° C. to 10° C., 20° C. to 40° C., 20° C. to 30° C.). In some embodiments, condensing an N-terminal amino group of a C-protected amino acid or a C-protected peptide with an N-Boc amino acid or an N-Boc peptide occurs at a temperature in the range of 0° C. to 40° C. In some embodiments, the condensing occurs at a temperature in the range of 10° C. to 40° C. In some embodiments, the condensing occurs at a temperature in the range of 20° C. to 40° C. In some embodiments, the condensing occurs at a temperature in the range of 20° C. to 30° C. In some embodiments, the condensing occurs at a temperature of 25° C.

Each condensation reaction can proceed until a desired level of conversion is achieved. For example, in some embodiments, the (i) condensing and the (iii) condensing can each independently proceed for a time in the range of 1 hour to 72 hours, such as, e.g., 1 hour to 30 hours, 2 hours to 24 hours, 12 hours to 24 hours, or overnight.

Following each condensation reaction, the reaction mixture can be washed with an aqueous solution, such as a brine solution, one or more times to separate an organic layer comprising an N-Boc C-protected peptide from an aqueous layer comprising unreacted reagents and N-Boc amino acids or N-Boc peptides. In some embodiments, the reaction mixture is washed once. In some embodiments, the reaction mixture is washed once with a brine solution.

In some embodiments, between the (i) condensing and the (ii) removing, the method further comprises (ia) washing the first reaction mixture with a first aqueous solution, such as a brine solution, and separating a first organic layer comprising the first N-Boc C-protected peptide.

In some embodiments, between the (iii) condensing and the (iv) removing, the method further comprises (iiia) washing the third reaction mixture with a second aqueous solution, such as a brine solution, and separating a third organic layer comprising the second N-Boc C-protected peptide.

Alternatively, N-Boc C-protected peptides can be isolated by precipitation in a polar solvent system.

For example, in some embodiments, between the (i) condensing and the (ii) removing, the method further comprises (ia′) precipitating the first N-Boc C-protected peptide in a first polar solvent system and isolating the first N-Boc C-protected peptide by solid-liquid separation. In some embodiments, the first polar solvent system is selected from water, methanol (MeOH), ethanol (EtOH), isopropyl alcohol (IPA), acetic acid (AcOH), acetonitrile (ACN), acetone, isopropyl acetate (iPrOAc), N-methyl-2-pyrrolidone (NMP), dimethyl sulfoxide (DMSO), dimethylformamide (DMF), heptanes, dichloromethane (DCM), cyclopentyl methyl ether (CPME), and combinations of any of the foregoing. In some embodiments, the first polar solvent system comprises a water-acetonitrile mixture. In other embodiments, the first polar solvent system consists of acetonitrile.

Similarly, in some embodiments, between the (iii) condensing and the (iv) removing, the method further comprises (iiia′) precipitating the second N-Boc C-protected peptide in a third polar solvent system and isolating the second N-Boc C-protected peptide by solid-liquid separation. In some embodiments, the third polar solvent system is selected from water, MeOH, EtOH, IPA, AcOH, ACN, acetone, iPrOAc, NMP, DMSO, DMF, heptanes, DCM, CPME, and combinations of any of the foregoing. In some embodiments, the third polar solvent system comprises a water-acetonitrile mixture. In other embodiments, the third polar solvent system consists of acetonitrile.

The (ii) removing can be performed under similar conditions to the (iv) removing (e.g., reagent(s), solvent(s), reaction time(s) and temperature(s)); however, the (ii) and (iv) anchor group removal conditions can be independently selected.

In some embodiments, the (ii) removing comprises removing the first benzyl alcohol anchor group from the first N-Boc C-protected peptide to obtain a second reaction mixture comprising the second N-Boc peptide. Additionally, in some embodiments, the (iv) removing comprises removing the second benzyl alcohol anchor group from the second N-Boc C-protected peptide to obtain a fourth reaction mixture comprising the third N-Boc peptide.

In some embodiments, the first benzyl alcohol anchor group and/or the second benzyl alcohol anchor group is removed by base-catalyzed ester hydrolysis. In some embodiments, the base used for base-catalyzed ester hydrolysis is selected from sodium hydroxide, potassium hydroxide, lithium hydroxide, barium hydroxide, and tetrabutylammonium hydroxide. In some embodiments, the base used for base-catalyzed ester hydrolysis is sodium hydroxide. In some embodiments, the base used for base-catalyzed ester hydrolysis is potassium hydroxide. In some embodiments, the base used for base-catalyzed ester hydrolysis is lithium hydroxide. In some embodiments, the base used for base-catalyzed ester hydrolysis is barium hydroxide. In some embodiments, the base used for base-catalyzed ester hydrolysis is tetrabutylammonium hydroxide. The base used for base-catalyzed ester hydrolysis can be independently selected for the (ii) removing and the (iv) removing.

In some embodiments, the base-catalyzed ester hydrolysis is performed in a solvent system comprising water and THF. In some embodiments, the volumetric ratio of water to THF in the solvent system is 1:4. The solvent system used for base-catalyzed ester hydrolysis can be independently selected for the (ii) removing and the (iv) removing.

In some embodiments, the base used for base-catalyzed ester hydrolysis is lithium hydroxide, and the base-catalyzed ester hydrolysis is performed in a solvent system comprising water and THF. In some embodiments, the base used for base-catalyzed ester hydrolysis is lithium hydroxide, and the base-catalyzed ester hydrolysis is performed in a solvent system comprising water and THF at a 1:4 volumetric ratio.

In some embodiments, between the (ii) removing and the (iii) condensing, the method further comprises (iia) acidifying and diluting the second reaction mixture comprising the second N-Boc peptide. In some embodiments, the acidifying comprises adding an acid to the second reaction mixture, wherein the acid is selected from methanesulfonic acid, trifluoromethanesulfonic acid, benzenesulfonic acid, p-toluenesulfonic anhydride, sulfuric acid, and hydrogen chloride. In some embodiments, the acid added is hydrogen chloride. In some embodiments, the diluting comprises diluting the second reaction mixture with an organic solvent selected from ethanol, ethyl acetate, toluene, isopropanol, acetonitrile, methanol, hexane, dimethyl sulfoxide, tetrahydrofuran, acetone, dichloromethane, dimethylformamide, and cyclohexane. In some embodiments, the organic solvent used for dilution is ethyl acetate.

Additionally, in some embodiments, between the (iia) acidifying and diluting and the (iii) condensing, the method further comprises (iib) separating a second organic layer comprising the second N-Boc peptide and concentrating the second organic layer to obtain a first concentrated mixture comprising the second N-Boc peptide. In some embodiments, between the (iib) separating and concentrating and the (iii) condensing, the method further comprises (iic) dissolving the first concentrated mixture in a first ethereal solvent, precipitating the first benzyl alcohol anchor group in a second polar solvent system, and removing the first benzyl alcohol anchor group by solid-liquid separation to obtain a first filtrate comprising the second N-Boc peptide.

In some embodiments, the ethereal solvent is selected from dimethyl ether, tetrahydrofuran, 2-MeTHF, cyclopentyl methyl ether, and 1,4-dioxane. In some embodiments, the ethereal solvent is tetrahydrofuran.

In some embodiments, the second polar solvent system is selected from water, MeOH, EtOH, IPA, AcOH, ACN, acetone, iPrOAc, NMP, DMSO, DMF, heptanes, DCM, CPME, and combinations of any of the foregoing. In some embodiments, the second polar solvent system comprises a water-acetonitrile mixture. In other embodiments, the second polar solvent system consists of acetonitrile.

In addition, in some embodiments, between the (iic) dissolving and the (iii) condensing, the method further comprises (iid) concentrating the first filtrate to isolate the second N-Boc peptide.

The steps of (iii) condensing and (iv) removing may be repeated for one or more cycles to assemble additional C-protected fragments into a longer peptide. For example, in some methods of the present disclosure, multiple cycles of convergent peptide assembly can be performed by repeating the general steps of condensation and anchor group removal, where the specific conditions for each step in each cycle (e.g., reaction temperature, reaction time, choice and ratio of reagents) can be independently selected.

Illustratively, in some embodiments, the methods further comprise repeating the (iii) condensing and the (iv) removing for one or more cycles, wherein:
the third N-Boc peptide from the (iv) removing in a cycle becomes the second N-Boc peptide in the (iii) condensing of the following cycle; and
the second C-protected peptide of the (iii) condensing of a cycle may be the same as or different from the second C-protected peptide of the (iii) condensing of the preceding cycle.

Between the (iv) removing of one cycle and the (iii) condensing of the next cycle, an N-Boc peptide may be isolated by steps analogous to the (iia), (iib), (iic), and (iid) above.

Following the final condensation step, the final N-Boc C-protected peptide can be globally deprotected by a variety of means, such as, e.g., by acid hydrolysis, to obtain the assembled peptide. Global deprotection methods using, for example, an acid (e.g., trifluoroacetic acid (TFA)) or a fluorine-substituted alcohol (e.g., trifluoroethanol (TFE) or hexafluoroisopropanol (HFIP)) are described above. The assembled peptide can then be isolated and purified according to known techniques of peptide chemistry. For example, the reaction mixture can be subjected to extraction washing and crystallization to isolate and purify the peptide.

### Continuous Manufacturing

Flow synthesis can shorten synthesis times and ease scale-up of synthetic methods, including peptide synthesis methods. In some embodiments of the present disclosure, the liquid phase peptide synthesis methods described herein can be implemented in a flow reactor. Flow reactors for use in synthetic methods, including peptide synthesis methods, are known in the art.

The following examples are given for the purpose of illustrating various embodiments of the disclosure and are not meant to limit the present disclosure in any fashion. One skilled in the art will appreciate readily that the present disclosure is well-adapted to carry out the objects and obtain the ends and advantages mentioned, as well as those objects, ends, and advantages inherent herein. Changes therein and other uses which are encompassed within the spirit of the disclosure as defined by the scope of the claims will occur to those skilled in the art.

### EXAMPLES

This section provides specific examples of liquid phase peptide synthesis methods described herein, including convergent synthesis methods described in this application.

TABLE 1
List of Abbreviations

| % | percent |
|---|---|
| 2-MeTHF | 2-methyltetrahydrofuran |
| AA | amino acid |
| ACN or MeCN | acetonitrile |
| AcOH | acetic acid |
| AIB | 2-aminoisobutyric acid |
| aq or aq. | aqueous |
| CPME | cyclopentyl methyl ether |
| DBU | 1,8-diazabicyclo[5.4.0]undec-7-ene |
| DCM | dichloromethane |
| DMAP | N,N-dimethylpyridin-4-amine |
| DMF | N,N-dimethylformamide |
| DMSO | dimethyl sulfoxide |
| EDC | 1-ethyl-3-(3-dimethylaminopropyl)carbodiimide |
| eq or eq. or equiv. | equivalent |
| EtOAc | ethyl acetate |
| EtOH | ethanol |
| Fmoc | 9-fluorenylmethoxycarbonyl |
| g or gr | gram(s) |
| Glu | glutamic acid |
| Gly | glycine |
| h | hour(s) |
| H₂O | water |
| HCl | hydrochloric acid |
| His | histidine |
| HOBt | 1-hydroxybenzotriazole |
| HPLC | high pressure liquid chromatography |
| iPrOH or IPA | iso-propanol |
| iPrOAc | isopropyl acetate |
| L | liter(s) |
| LC | liquid chromatography |
| LiOH | lithium hydroxide |
| Lys | lysine |
| M | molar |
| Me | methyl |
| MeOH | methanol |
| mg | milligram(s) |
| min | minute(s) |
| mL | milliliter(s) |
| MTBE | methyl tert-butyl ether |
| NaCl | sodium chloride |
| Na₂CO₃ | sodium carbonate |
| NMP | N-methyl-2-pyrrolidinone |
| NMR | nuclear magnetic resonance |
| PhMe | toluene |
| RT or rt or r.t. | room temperature |
| Ser | serine |
| tBu | tert-butyl |
| THF | tetrahydrofuran |
| Trt | trityl |
| V or vol or vol. | volume(s) |

Provided in this section are descriptions of the general analytical methods used to prepare the specific examples provided herein.

Proton NMR Spectra: Unless otherwise indicated, all ¹H NMR spectra were collected on a Bruker NMR instrument at 400 or 500 MHz. All observed protons are reported as parts-per-million (ppm) downfield from tetramethylsilane (TMS) using the internal solvent peak as reference. Some 1H signals may be missing due to exchange with D from CD3OD, or due to signal suppression.

### Section 1: Generic Liquid Phase Peptide Synthesis Route

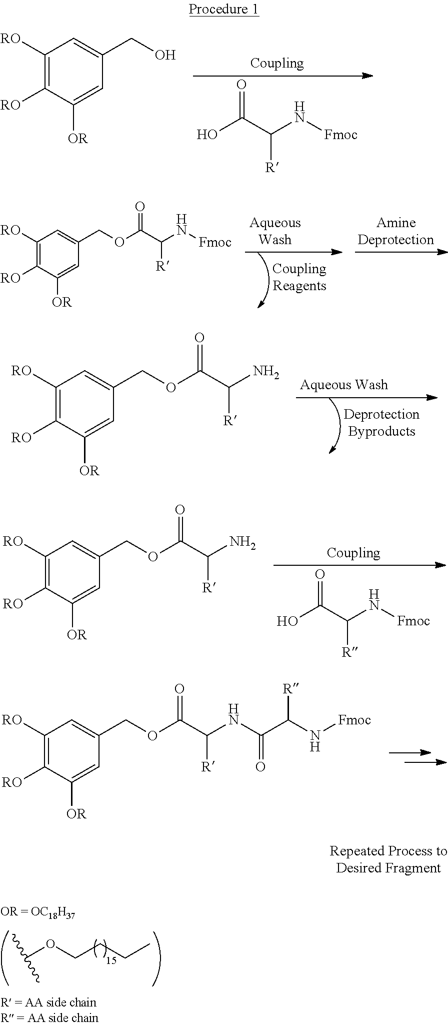

Provided in this section is a generic protocol (Procedure 1) for performing a liquid phase peptide synthesis process of the present disclosure using a C18 tag (“C18 TAG”).

An overhead stirring reactor is charged with (3,4,5-trioctadecoxyphenyl)methanol (limiting reagent) and 2-methyltetrahydrofuran (10-20 L/kg). Fmoc-protected amino acid (1.2-1.6 equiv.) is then added to the reactor. 4-dimethylaminopyridine (0.2 equiv.) and 1-(3-dimethylaminopropyl)-3-ethylcarbodiimide hydrochloride (1.6 equiv.) are added to the reactor in one portion, respectively. The reaction is stirred at 25° C. for approximately 15-16 hours. Following reaction completion, aqueous 20% NaCl solution (6 L/kg) is added to the reactor and the biphasic system is agitated for 15 minutes. Once agitation is stopped and a clean phase separation is formed, the lower aqueous layer is cut out. Mercaptosuccinic acid (3.0 equiv.) is added to the reactor in one portion followed by 1,8-diazabicyclo[5.4.0]undec-7-ene (7.0 equiv.) via dropwise addition over the course of 10 minutes. The reaction is stirred for 20-25 minutes at 25° C. Following complete Fmoc deprotection, aqueous 10% sodium carbonate:DMF (5:1) solution* (6 L/kg) is added to the reactor and agitated for approximately 20 minutes. Once agitation is stopped and a clean phase cut is formed, the lower aqueous layer is cut out. The same washing procedure is performed for an addition of an aqueous 20% NaCl solution (6 L/kg), which is sequentially added to the reactor. Following removal of the lower aqueous layer, acetonitrile (12 L/kg) is added over the course of 1 hour resulting in a viscous slurry. The reactor jacket temperature is set to 40° C. and held with agitation for 30 minutes. Setting a slow cooldown to 25° C. over the course of 15 minutes provides a slurry with significantly lowered viscosity. The reaction slurry is drained into a collection vessel and sequentially filtered. The resulting cake is washed with acetonitrile (12.5 L/kg) and dried under a stream of nitrogen with light vacuum applied to provide the desired product.

Solely 10% sodium carbonate washes can be used following Fmoc deprotection without utilizing a co-solvent system with DMF. Each wash system is applicable within this chemistry.

Examples of peptides that have been produced by following methods substantially similar to Procedure 1, using appropriate starting materials, include C18 TAG-Lys(Trt)—NH₂ (90% yield); C18 TAG-Lys(Trt)-Ser(′Bu)—NH₂*, and C18 TAG-Lys(Trt)-Ser(′Bu)-Gly-Gly-NH₂*. A “*” indicates a peptide was made via continuous solution LPPS, but the reaction stream was carried forward and an isolated yield was not obtained/applicable.

### Section 2: C18 Tag Solubility

Provided in this section is an analysis of the solubility of the C18 tag in various organic solvents. All starting materials are either commercially available from Sigma-Aldrich, Oakwood Chemicals, ChemPep Inc., CombiBlocks, or similar vendors, unless otherwise noted, or known in the art and may be synthesized by employing known procedures using ordinary skill.

(3,4,5-trioctadecoxyphenyl)methanol (200 mg) was measured out into a series of 1-dram vials. 1 mL of a screening solvent was added to its corresponding 1-dram vial. The slurry vials were placed on a shaker plate set to 20° C. and agitated overnight. Following agitation, the slurries were transferred to centrifuge tubes and the supernatant was isolated. 100 μL of the isolated supernatant was diluted in 10 mL THF to provides solutions for HPLC analysis using the Agilent 1290 Infinity II system with a Cortecs UPLC C8 1.6 μm 2, 1×150 mm column with a dilution factor of 100. A typical run through the HPLC instrument used a column temperature of 30° C.; an injection volume of 1 μL; a method length of 15 min; a flow rate of 0.3 mL/min; UV Detection: 220 nm; Mobile Phase A: 0.1% TFA in water; Mobile Phase B: 0.1% TFA in ACN; HPLC Gradient: 0 min—95:5 (A:B), 10 min—5:95 (A:B), 13 min—95:5 (A:B). The sample solutions were assayed on a calibrated LC system, and the corresponding C18 tag concentrations are reported below.

TABLE 2
C18 Tag Solubility

| SOLVENT | TEMPERATURE (° C.) | SOLUBILITY | |
|---|---|---|---|
| Water | 20 | 0 | mg/mL |
| MeOH | 20 | 0 | mg/mL |
| EtOH | 20 | 0 | mg/mL |
| IPA | 20 | 0 | mg/mL |
| AcOH | 20 | 0 | mg/mL |
| ACN | 20 | 0 | mg/mL |
| Acetone | 20 | 0 | mg/mL |
| iPrOAc | 20 | 1.81 | mg/mL |
| THF | 20 | ≥200 | mg/mL |
| MTBE | 20 | 43.83 | mg/mL |
| NMP | 20 | 0 | mg/mL |
| DMSO | 20 | 0 | mg/mL |
| DMF | 20 | 0 | mg/mL |
| PhMe | 20 | 57.82 | mg/mL |
| Heptanes | 20 | 1.63 | mg/mL |
| 2-Me-THF | 20 | 172.40 | mg/mL |
| 1:1 THF:Water | 20 | 0 | mg/mL |
| 1:2 IPA:Water | 20 | 0 | mg/mL |
| 2:1 ACN:Water | 20 | 0 | mg/mL |
| DCM | 20 | 0 | mg/mL |
| CPME | 20 | 6.80 | mg/mL |

### Section 3: Synthesis of Example C-Protected Peptides

### Example 1-001: (3,4,5-trioctadecoxyphenyl)methyl 2-amino-6-(tritylamino)hexanoate

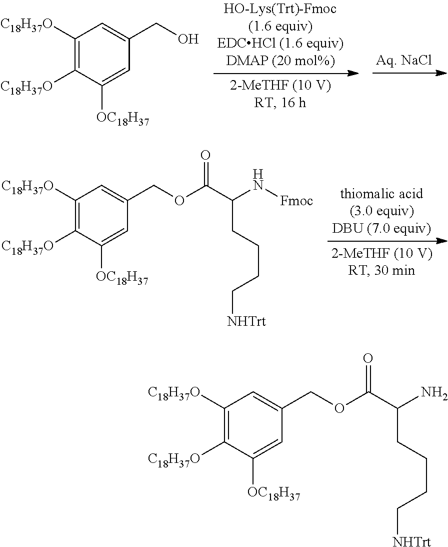

To a 500 mL overhead stirring reactor was charged 2-methyltetrahydrofuran (200.0 mL, 1995 mmol, 100 mass %) and (3,4,5-trioctadecoxyphenyl)methanol (20.01 g, 21.90 mmol, 100 mass %). 2-(9H-fluoren-9-ylmethoxycarbonylamino)-6-(tritylamino)hexanoic acid (1.6 equiv., 35.04 mmol, 100 mass %) was then added to the reactor. 4-dimethylaminopyridine (0.2 equiv., 4.380 mmol, 97 mass %) and 1-(3-dimethylaminopropyl)-3-ethylcarbodiimide hydrochloride (1.6 equiv., 35.04 mmol, 100 mass %) were then added to the reactor in one portion, respectively. The reaction was stirred at 25° C. for approximately 15-16 hours. Following reaction completion, an aqueous 20% NaCl solution (120 mL, 6 volumes) was added to the reactor, and the biphasic system was agitated for 15 minutes. Once agitation was stopped and a clean phase separation had formed, the lower aqueous layer was cut out. Thiomalic acid (3.0 equiv., 65.71 mmol, 100 mass %) was added to the reactor in one portion followed by 1,8-diazabicyclo[5.4.0]undec-7-ene (7.0 equiv., 153.3 mmol, 100 mass %) via dropwise addition over the course of 10 minutes. The reaction was stirred for 20-25 minutes at 25° C. Following confirmation of complete Fmoc deprotection, an aqueous 10% sodium carbonate:DMF (5:1) solution (120 mL, 6 volumes) was added to the reactor, and the resulting system was agitated for approximately 20 minutes. Once agitation was stopped and a clean phase cut had formed, the lower aqueous layer was cut out. The same washing procedure was performed for an addition of an aqueous 20% NaCl solution (120 mL, 6 volumes), which was sequentially added to the reactor. Following removal of the lower aqueous layer, acetonitrile (240 mL, 12 volumes) was added over the course of 1 hour, resulting in a viscous slurry. The reactor jacket temperature was set to 40° C., where it was observed that the slurry returned to a homogenous mixture. Setting a slow cooldown to 25° C. over the course of 15 minutes provided a slurry with significantly lowered viscosity. The reaction slurry was drained into a 1 L collection bottle and sequentially filtered. The resulting cake was washed with acetonitrile (250 mL, 12.5 volumes) and dried under a stream of nitrogen with light vacuum applied to provide (3,4,5-trioctadecoxyphenyl)methyl 2-amino-6-(tritylamino)hexanoate (25.40 g, 19.78 mmol, 90.3% yield).

¹H NMR (400 MHz, CHLOROFORM-d) δ ppm 7.49 (d, J=7.46 Hz, 6H), 7.25-7.31 (m, 9H), 7.17-7.22 (m, 2H), 6.53 (s, 2H), 5.05-5.13 (m, 1H), 4.98-5.03 (m, 1H), 3.92-3.98 (m, 6H), 3.48-3.52 (m, 1H), 2.17 (br t, J=6.01 Hz, 2H), 1.71-1.85 (m, 9H), 1.39-1.60 (m, 12H), 1.32-1.34 (m, 6H), 1.28 (s, 71H), 1.19-1.38 (m, 1H), 0.85-0.92 (m, 9H).

### Example 1-002: 3,4,5-tris(octadecyloxy)benzyl N2-(O-(tert-butyl)seryl)-N6-trityllysinate (C18 TAG-Lys(Trt)-Ser(tBu)—NH₂)

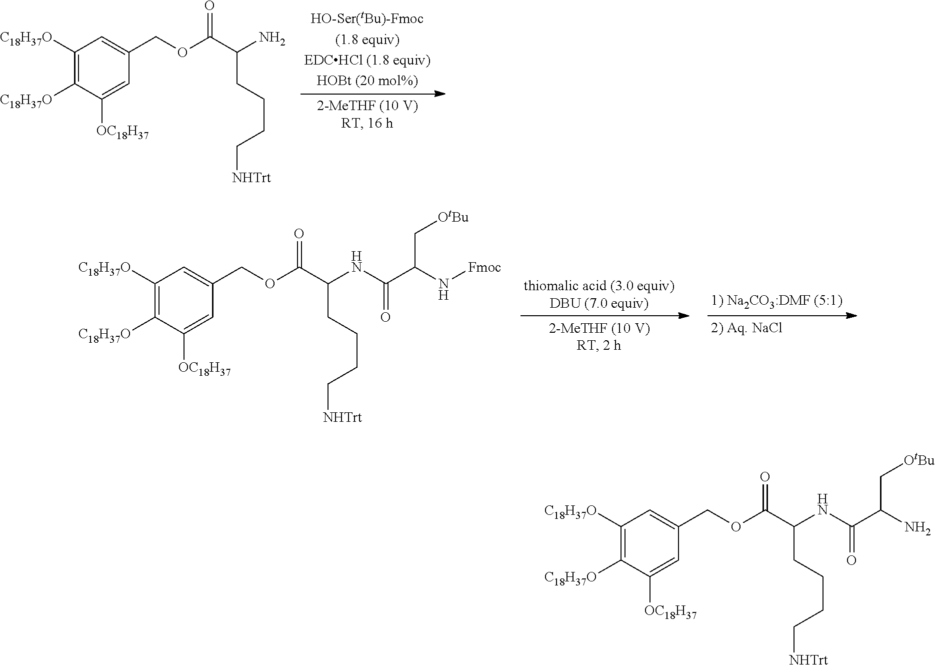

An overhead stirring 500 mL Chemglass reactor was charged with 2-methyltetrahydrofuran (245.4 mL, 2447 mmol, 100 mass %). The reactor jacket temperature was set to 25° C. (3,4,5-trioctadecoxyphenyl)methyl 2-amino-6-(tritylamino)hexanoate (C, 24.54 g, 19.11 mmol, 100 mass %) was added to the reactor in one portion. N-Fmoc-O-tert-butyl-L-serine (1.6 equiv., 30.58 mmol, 95 mass %) was added to the reactor. 1-hydroxybenzotriazole (0.2 equiv., 3.822 mmol, 98 mass %) was added in one portion to the reactor. 1-(3-dimethylaminopropyl)-3-ethylcarbodiimide hydrochloride (1.6 equiv., 30.58 mmol, 100 mass %) was added to the reactor. The reaction was allowed to stir until full consumption of the starting material was observed. Following confirmation of reaction completion, a 20% NaCl solution (150 mL, 6.1 volumes) was added to the reactor, and the biphasic system was agitated for 10 minutes. Stirring was stopped and a clean phase split formed shortly after. The lower aqueous layer was then cut out. Thiomalic acid (3.0 equiv., 57.33 mmol, 99 mass %) was added to the reactor in one portion. 1,8-diazabicyclo[5.4.0]undec-7-ene (7.0 equiv., 133.8 mmol, 100 mass %) was added to the reactor via syringe. The Fmoc deprotection was allowed to stir for 15 minutes prior to sampling for reaction completion. Conversion was monitored by liquid chromatography and verified against a starting material reference standard. Once full Fmoc deprotection was confirmed, a 10% sodium carbonate:DMF (5:1) solution (150 mL 6.1 volumes) was added to the reactor. The biphasic system was agitated for 15 minutes. Stirring was stopped to provide a clean phase cut, and the lower aqueous layer was cut out. The same washing procedure was performed for an addition of an aqueous 20% NaCl solution (150 mL, 6.1 volumes), which was sequentially added to the reactor. Following the removal of the lower aqueous layer, the isolated organic solution was carried forward into a sequential HO-Gly-Gly-Fmoc coupling following a similar procedure.

¹H NMR (400 MHz, CHLOROFORM-d) δ ppm 7.46 (br d, J=7.46 Hz, 6H), 7.25-7.30 (m, 9H), 7.16-7.21 (m, 2H), 6.58 (s, 2H), 4.94-5.12 (m, 1H), 4.61 (br d, J=4.15 Hz, 2H), 3.92-4.02 (m, 8H), 1.63-1.94 (m, 12H), 1.42-1.54 (m, 9H), 1.28 (s, 86H), 1.26-1.26 (m, 1H), 1.25-1.32 (m, 1H), 1.11-1.23 (m, 9H), 0.87-0.94 (m, 9H).

### Example 1-003: 3,4,5-tris(octadecyloxy)benzyl N2-(O-(tert-butyl)-N-glycylglycylseryl)-N6-trityllysinate (C18 TAG-Lys(Trt)-Ser(tBu)-Gly-Gly-NH₂)

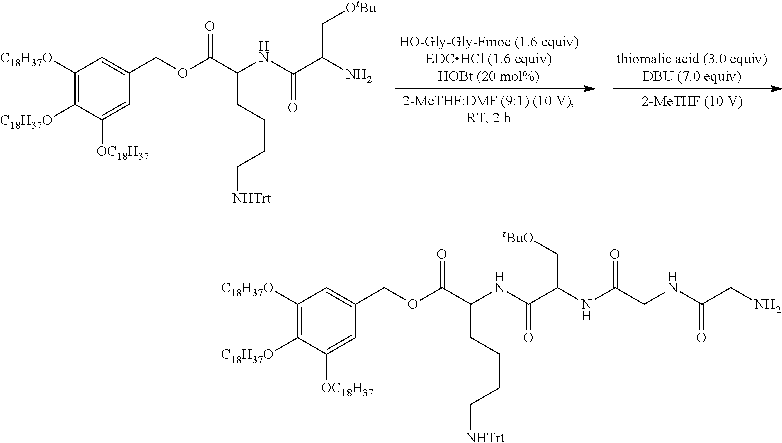

A 500 mL Chemglass reactor loaded with approximately 230 mL 2-MeTHF solution containing C18 TAG-Lys-Ser-NH₂ in approximately 90 mg/mL concentration was utilized for continuous solution chemistry. N,N-dimethylformamide (30 mL, 387.855 mmol, 100 mass %) was added to the reactor to provide an approximately 9:1 co-solvent system of 2-MeTHF:DMF. Fmoc-Gly-Gly-OH (1.6 equiv., 28.03 mmol, 99 mass %) was added to the reactor. 1-hydroxybenzotriazole (0.2 equiv., 3.503 mmol, 98 mass %) was added to the reactor. 1-(3-dimethylaminopropyl)-3-ethylcarbodiimide hydrochloride (1.6 equiv., 28.03 mmol, 100 mass %) was added to the reactor. Full conversion of starting material was observed in approximately 2 hours. Following confirmation of reaction completion, a 20% NaCl solution (170 mL, 6.8 volumes) was added to the reactor, and the biphasic system was agitated for 10 minutes. Once agitation was stopped and a clean phase separation had formed, the lower aqueous layer was cut out. Thiomalic acid (3.0 equiv., 52.55 mmol, 100 mass %) was added to the reactor in one portion. 1,8-diazabicyclo[5.4.0]undec-7-ene (7.0 equiv., 122.6 mmol, 100 mass %) was added to the reactor via syringe. The reaction was stirred for approximately 15 minutes prior to sampling to confirm Fmoc deprotection completion. Once Fmoc deprotection completion was confirmed, a 10% sodium carbonate solution (170 mL, 6.8 volumes) was added to the reactor, and the biphasic system was agitated for 10 minutes. Once agitation was stopped and a clean phase separation had formed, the lower aqueous layer was cut out. The same washing procedure was performed for an addition of an aqueous 20% NaCl solution (170 mL, 6.8 volumes), which was sequentially added to the reactor. Following the removal of the lower aqueous layer, the isolated organic solution was carried forward into a sequential HO-Gly-Gly-Fmoc coupling following a similar procedure.

¹H NMR (400 MHz, CHLOROFORM-d) δ ppm 7.43-7.50 (m, 6H), 7.24-7.31 (m, 9H), 7.15-7.23 (m, 2H), 6.51 (s, 2H), 5.06-5.15 (m, 1H), 4.97-5.04 (m, 1H), 4.61 (td, J=7.77, 4.98 Hz, 1H), 3.89-4.05 (m, 8H), 3.88-4.02 (m, 1H), 3.83 (dd, J=8.50, 3.73 Hz, 1H), 3.29-3.42 (m, 3H), 3.17 (br s, 8H), 2.11 (br s, 2H), 2.02 (s, 2H), 1.67-1.88 (m, 8H), 1.40-1.54 (m, 11H), 1.32-1.34 (m, 6H), 1.28 (s, 72H), 1.17-1.21 (m, 1H), 1.10-1.24 (m, 9H), 0.84-0.96 (m, 9H).

### Section 4: Example Convergent Synthesis Methods

### Example 2-001: tert-butyl (4S)-4-[(2-amino-2-methyl-propanoyl)amino]-5-oxo-5-[[2-oxo-2-[(3,4,5-trioctadecoxyphenyl)methoxy]ethyl]amino]pentanoate

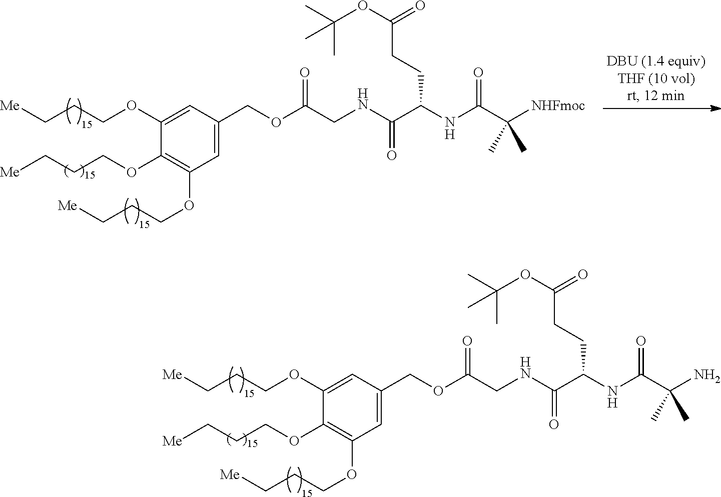

A 150 mL round bottom flask equipped with a stir bar was charged with tert-butyl (4S)-4-[[2-(9H-fluoren-9-ylmethoxycarbonylamino)-2-methyl-propanoyl]amino]-5-oxo-5-[[2-oxo-2-[(3,4,5-trioctadecoxyphenyl)methoxy]ethyl]amino]pentanoate (1.600 g, 0.9903 mmol, 90.56 mass %), followed by tetrahydrofuran (16 mL, 10 vol). 1,8-diazabicyclo[5.4.0]undec-7-ene (0.23 mL, 1.386 mmol, 1.4 equiv) was added to the reaction at room temperature. After 12 minutes, aqueous 6 M HCl (0.28 mL, 1.68 mmol) was added to the reaction mixture, followed by acetonitrile (20 mL). A white precipitate formed, which was filtered and washed 3 times with acetonitrile (3×20 mL). The white solid was dried on the fritted glass filter, then transferred to a 150 mL round bottom flask and dried under low vacuum. 1.528 g (0.9098 mmol, 92% yield) of tert-butyl (4S)-4-[(2-amino-2-methyl-propanoyl)amino]-5-oxo-5-[[2-oxo-2-[(3,4,5-trioctadecoxyphenyl)methoxy]ethyl]amino]pentanoate was obtained as a white solid with 73.84% potency.

¹H NMR (400 MHz, CHLOROFORM-d) δ ppm 6.53-6.55 (m, 1H), 5.06 (s, 1H), 3.93-4.01 (m, 4H), 2.02 (s, 9H), 1.73-1.88 (m, 4H), 1.35-1.51 (m, 15H), 1.28 (s, 50H), 0.86-0.93 (m, 6H), 0.09 (s, 1H).

### Example 2-002: tert-butyl (4S)-4-[[2-[[(2S)-2-(tert-butoxycarbonylamino)-3-(1-tritylimidazol-4-yl)propanoyl]amino]-2-methyl-propanoyl]amino]-5-oxo-5-[[2-oxo-2-[(3,4,5-trioctadecoxyphenyl)methoxy]ethyl]amino]pentanoate

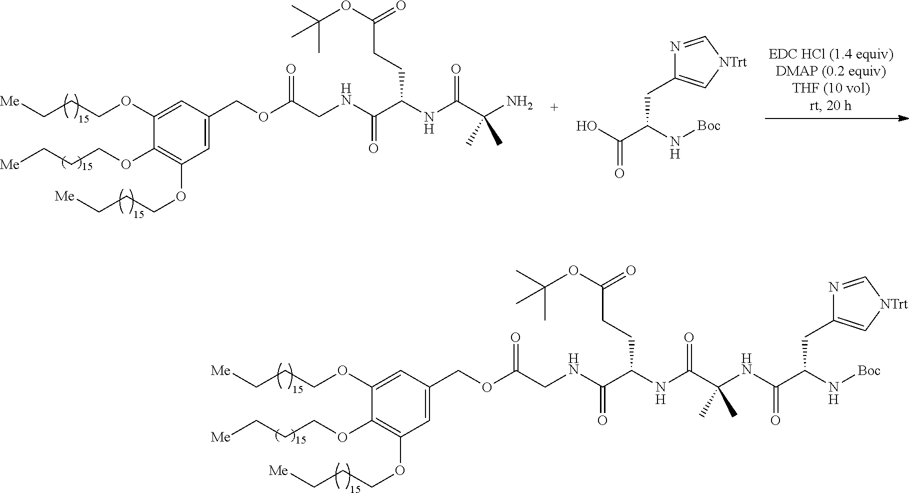

A 150 mL round bottom flask equipped with a stir bar was placed under an atmosphere of nitrogen and charged with tert-butyl (4S)-4-[(2-amino-2-methyl-propanoyl)amino]-5-oxo-5-[[2-oxo-2-[(3,4,5-trioctadecoxyphenyl)methoxy]ethyl]amino]pentanoate (1.451 g, 0.8634 mmol, 73.84 mass %). Tetrahydrofuran (14 mL, 10 vol) was added, followed by Boc-His(Trt)-OH (0.6137 g, 1.209 mmol, 1.4 equiv), 1-(3-dimethylaminopropyl)-3-ethylcarbodiimide hydrochloride (0.2317 g, 1.209 mmol, 1.4 equiv), and 4-dimethylaminopyridine (21 mg, 0.1727 mmol, 0.2 equiv). After stirring under nitrogen at ambient temperature for 20 h, acetonitrile (20 mL) was added to the reaction mixture to precipitate the product. A white precipitate formed, which was filtered and washed 3 times with acetonitrile (3×20 mL). The white solid was dried on the fritted glass filter, then transferred to a 150 mL round bottom flask and dried under low vacuum. 1.482 g (0.8110 mmol, 94% yield) of tert-butyl (4S)-4-[[2-[[(2S)-2-(tert-butoxycarbonylamino)-3-(1-tritylimidazol-4-yl)propanoyl]amino]-2-methyl-propanoyl]amino]-5-oxo-5-[[2-oxo-2-[(3,4,5-trioctadecoxyphenyl)methoxy]ethyl]amino]pentanoate was obtained as a white solid with 94.15% potency.

¹H NMR (400 MHz, CHLOROFORM-d) δ ppm 7.63 (br s, 1H), 7.49 (br s, 1H), 7.32-7.39 (m, 5H), 7.09-7.17 (m, 3H), 6.75 (s, 1H), 6.53 (s, 1H), 4.94-5.09 (m, 1H), 3.90-4.01 (m, 3H), 3.01 (br s, 1H), 2.24-2.41 (m, 1H), 2.02 (s, 3H), 1.67-1.89 (m, 4H), 1.63 (br s, 1H), 1.44-1.52 (m, 9H), 1.38-1.40 (m, 6H), 1.28 (s, 43H), 0.85-0.95 (m, 5H).

### Example 2-003: Boc-His(Trt)-AIB-Glu(OtBu)-Gly-OH

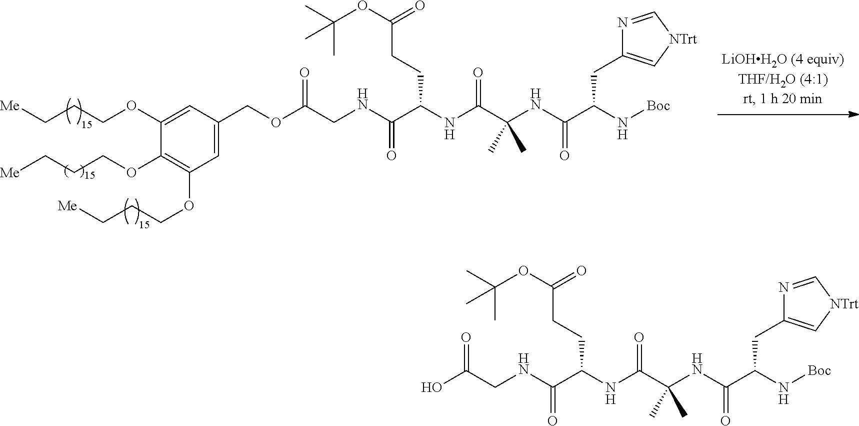

A 150 mL round bottom flask equipped with a stir bar was charged with tert-butyl (4S)-4-[[2-[[(2S)-2-(tert-butoxycarbonylamino)-3-(1-tritylimidazol-4-yl)propanoyl]amino]-2-methyl-propanoyl]amino]-5-oxo-5-[[2-oxo-2-[(3,4,5-trioctadecoxyphenyl)methoxy]ethyl]amino]pentanoate (1.326 g, 0.7256 mmol), followed by tetrahydrofuran (21.21 mL), water (5.3 mL), and lithium hydroxide monohydrate (0.1218 g, 2.902 mmol, 4 equiv). After stirring for 1 h 20 min at room temperature, the reaction was acidified with aqueous 1 M HCl (10 mL) and diluted with EtOAc (20 mL). The mixture was transferred to a separatory funnel, and the layers were separated. The aqueous layer was extracted with EtOAc (3×5 mL). The combined organics were dried over anhydrous sodium sulfate and concentrated in vacuo. An aliquot of tetrahydrofuran (2 mL) was used to dissolve the concentrate, and acetonitrile (20 mL) was added. A white precipitate formed, which was removed by filtration. The filtrate was concentrated, and 0.351 g (0.425 mmol, 58% yield) of Boc-His(Trt)-AIB-Glu(OtBu)-Gly-OH was obtained as a white solid.

¹H NMR (400 MHz, CHLOROFORM-d) δ ppm 8.23 (s, 2H), 7.97 (br s, 2H), 7.74 (br s, 2H), 7.63 (br s, 1H), 7.54 (s, 1H), 7.40-7.47 (m, 15H), 7.10-7.16 (m, 10H), 6.90 (br s, 1H), 6.54 (br s, 1H), 4.55 (br s, 1H), 4.39 (br s, 1H), 4.05-4.14 (m, 1H), 3.81 (br d, J=13.5 Hz, 1H), 3.44 (br d, J=15.5 Hz, 1H), 3.04-3.20 (m, 2H), 2.25-2.52 (m, 5H), 2.14-2.24 (m, 2H), 2.10 (s, 2H), 1.97-2.08 (m, 2H), 1.65 (s, 1H), 1.50 (s, 5H), 1.40-1.46 (m, 17H), 1.34-1.39 (m, 14H), 1.24-1.29 (m, 2H), 0.84-0.93 (m, 1H), 0.09 (s, 3H).

### Example 2-004: tert-butyl (4S)-5-[[2-[[(1S,2R)-1-[[(1S)-1-benzyl-2-[[(1S,2R)-2-tert-butoxy-1-[[(1S)-1-(tert-butoxymethyl)-2-oxo-2-[(3,4,5-trioctadecoxyphenyl)methoxy]ethyl]carbamoyl]propyl]amino]-2-oxo-ethyl]carbamoyl]-2-tert-butoxy-propyl]amino]-2-oxo-ethyl]amino]-4-[[2-[[(2S)-2-(tert-butoxycarbonylamino)-3-(1-tritylimidazol-4-yl)propanoyl]amino]-2-methyl-propanoyl]amino]-5-oxo-pentanoate

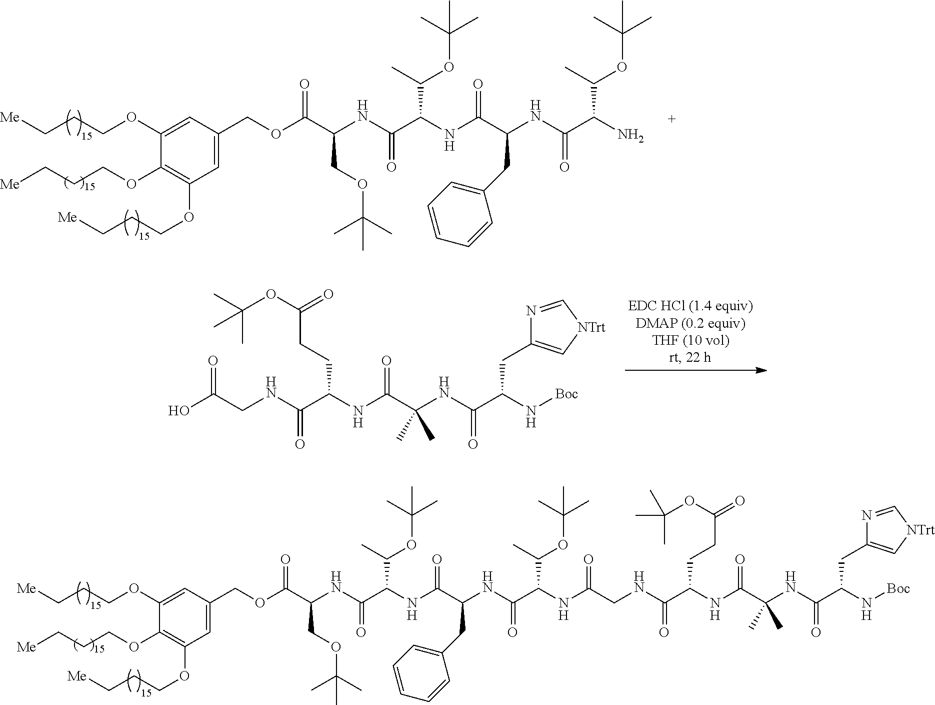

A 150 mL round bottom flask equipped with a stir bar was placed under an atmosphere of nitrogen and charged with (3,4,5-trioctadecoxyphenyl)methyl (2S)-2-[[(2S)-2-[[(2S)-2-[[(2S)-2-amino-3-tert-butoxy-butanoyl]amino]-3-phenyl-propanoyl]amino]-3-tert-butoxy-butanoyl]amino]-3-tert-butoxy-propanoate (0.460 g, 0.265 mmol, 87.46 mass %), followed by Boc-His(Trt)-AIB-Glu(OtBu)-Gly-OH (0.339 g, 0.371 mmol, 1.4 equiv). Tetrahydrofuran (4.60 mL, 10 vol) was added, followed by 1-(3-dimethylaminopropyl)-3-ethylcarbodiimide hydrochloride (0.071 g, 0.371 mmol, 1.4 equiv) and 4-dimethylaminopyridine (6 mg, 0.05 mmol, 0.2 equiv). After stirring under nitrogen at ambient temperature for 22 h, acetonitrile (20 mL) was added to the reaction mixture to precipitate the product. A white precipitate formed, which was filtered and washed 3 times with acetonitrile (3×20 mL). The white solid was dried on the fritted glass filter, then transferred to a 150 mL round bottom flask and dried under low vacuum. 0.377 g (0.145 mmol, 55% yield) of tert-butyl (4S)-5-[[2-[[(1S,2R)-1-[[(1S)-1-benzyl-2-[[(1S,2R)-2-tert-butoxy-1-[[(1S)-1-(tert-butoxymethyl)-2-oxo-2-[(3,4,5-trioctadecoxyphenyl)methoxy]ethyl]carbamoyl]propyl]amino]-2-oxo-ethyl]carbamoyl]-2-tert-butoxy-propyl]amino]-2-oxo-ethyl]amino]-4-[[2-[[(2S)-2-(tert-butoxycarbonylamino)-3-(1-tritylimidazol-4-yl)propanoyl]amino]-2-methyl-propanoyl]amino]-5-oxo-pentanoate was obtained as a white solid.

¹H NMR (500 MHz, CHLOROFORM-d) δ ppm 8.19 (d, J=7.3 Hz, 1H), 7.39-7.45 (m, 5H), 7.25-7.28 (m, 1H), 7.19-7.23 (m, 1H), 7.07-7.16 (m, 4H), 6.78 (d, J=7.3 Hz, 1H), 6.67 (br s, 1H), 5.32 (s, 1H), 3.92-3.97 (m, 2H), 3.50 (br s, 1H), 3.36 (br s, 3H), 3.27 (s, 2H), 3.14-3.25 (m, 6H), 2.85 (s, 7H), 2.32-2.39 (m, 1H), 2.29 (s, 1H), 1.99-2.07 (m, 3H), 1.72-1.82 (m, 2H), 1.24-1.45 (m, 42H), 1.13 (t, J=7.2 Hz, 4H), 0.99-1.08 (m, 5H), 0.90 (t, J=6.9 Hz, 3H).

### Example 2-005: (2S)-3-tert-butoxy-2-[[(2S,3R)-3-tert-butoxy-2-[[(2S)-2-[[(2S,3R)-3-tert-butoxy-2-[[2-[[(2S)-5-tert-butoxy-2-[[2-[[(2S)-2-(tert-butoxycarbonylamino)-3-(1-tritylimidazol-4-yl)propanoyl]amino]-2-methyl-propanoyl]amino]-5-oxo-pentanoyl]amino]acetyl]amino]butanoyl]amino]-3-phenyl-propanoyl]amino]butanoyl]amino]propanoic acid

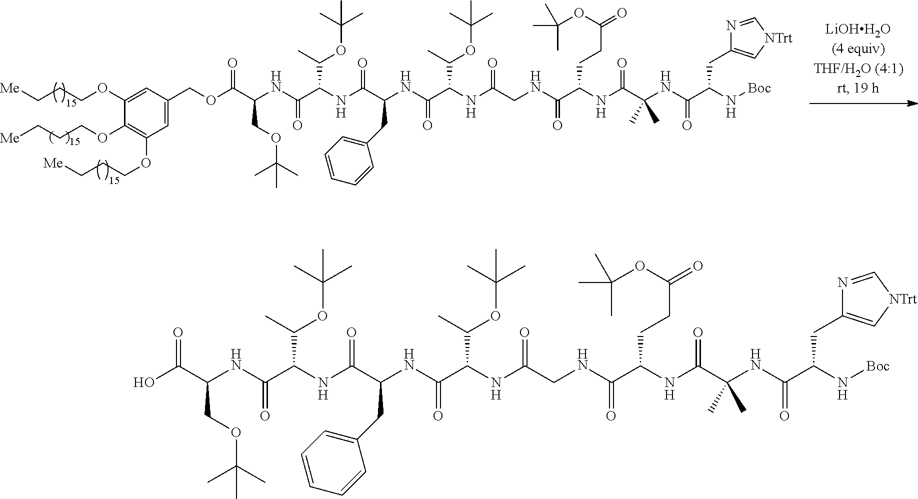

A 20 mL round bottom flask equipped with a stir bar was charged with tert-butyl (4S)-5-[[2-[[(1 S,2R)-1-[[(1 S)-1-benzyl-2-[[(1 S,2R)-2-tert-butoxy-1-[[(1 S)-1-(tert-butoxymethyl)-2-oxo-2-[(3,4,5-trioctadecoxyphenyl)methoxy]ethyl]carbamoyl]propyl]amino]-2-oxo-ethyl]carbamoyl]-2-tert-butoxy-propyl]amino]-2-oxo-ethyl]amino]-4-[[2-[[(2S)-2-(tert-butoxycarbonylamino)-3-(1-tritylimidazol-4-yl)propanoyl]amino]-2-methyl-propanoyl]amino]-5-oxo-pentanoate (0.100 g, 0.172 mmol), followed by tetrahydrofuran (1.6 mL), water (0.4 mL) and lithium hydroxide monohydrate (7 mg g, 0.17 mmol, 4 equiv). After stirring for 19 h at room temperature, the reaction was acidified with aqueous 1 M HCl (5 mL) and diluted with EtOAc (10 mL). The mixture was transferred to a separatory funnel and the layers were separated. The aqueous layer was extracted with EtOAc (3×5 mL). The combined organics were dried over anhydrous sodium sulfate and concentrated in vacuo. An aliquot of tetrahydrofuran (1 mL) was used to dissolve the concentrate, and acetonitrile (10 mL) was added. A white precipitate formed, which was removed by filtration. The filtrate was concentrated, and 56 mg (0.039 mmol, 91% yield) of (2S)-3-tert-butoxy-2-[[(2S,3R)-3-tert-butoxy-2-[[(2S)-2-[[(2S,3R)-3-tert-butoxy-2-[[2-[[(2S)-5-tert-butoxy-2-[[2-[[(2S)-2-(tert-butoxycarbonylamino)-3-(1-tritylimidazol-4-yl)propanoyl]amino]-2-methyl-propanoyl]amino]-5-oxo-pentanoyl]amino]acetyl]amino]butanoyl]amino]-3-phenyl-propanoyl]amino]butanoyl]amino]propanoic acid was obtained as a white solid.

1. A method of producing a peptide comprising:
(i) condensing an N-terminal amino group of a first C-protected amino acid or a first C-protected peptide with a C-terminal carboxy group of a first N-Fmoc amino acid or a first N-Fmoc peptide in the presence of a first coupling reagent in a first solvent system to obtain a first reaction mixture comprising a first N-Fmoc C-protected peptide, wherein:
the first solvent system comprises 2-methyltetrahydrofuran (2-MeTHF); and
a C-terminal carboxy group of the first C-protected amino acid or the first C-protected peptide is protected by an lipophilic molecular anchor group;
(ii) washing the first reaction mixture with a first aqueous solution and separating a first organic layer comprising the first N-Fmoc C-protected peptide;
(iii) removing an N-terminal Fmoc group from the first N-Fmoc C-protected peptide to obtain a second reaction mixture comprising a second C-protected peptide, wherein the removing is performed without an intervening isolation of the first N-Fmoc C-protected peptide from the first organic layer; and
(iv) washing the second reaction mixture with a second aqueous solution and separating a second organic layer comprising the second C-protected peptide.

2. The method of claim 1, wherein the lipophilic molecular anchor group is a benzyl alcohol anchor group.

3. The method of claim 1, wherein the lipophilic molecular anchor group is derived from 3,4,5-tri(octadecyloxy)benzyl alcohol.

4. The method of claim 1, wherein the first solvent system comprises 2-MeTHF at a concentration in the range of 70% (v/v) to 100% (v/v).

5. The method of claim 1, wherein the first solvent system further comprises dimethylformamide (DMF) or dimethylformamide (DMSO).

6. (canceled)

7. The method of claim 1, wherein:
the (ii) washing comprises washing the first reaction mixture with a saturated sodium chloride solution;
the (iii) removing comprises removing the N-terminal Fmoc group from the first N-Fmoc C-protected peptide with a first non-nucleophilic organic base in the presence of a first acidic thiol; and
the (iv) washing comprises a first wash with a second solvent system comprising sodium carbonate and optionally DMF and one or more optional additional washes with a saturated sodium chloride solution.

8. (canceled)

9. The method of claim 1, wherein the (iv) washing is performed without an intervening acid neutralization of the second reaction mixture.

10. (canceled)

11. The method of claim 7, wherein:
the first non-nucleophilic organic base is selected from N,N-diisopropylethylamine, 1,8-diazabicyclo[5.4.0]undec-7-ene (DBU), 1,4-diazabicyclo[2.2.2]octane (DABCO), and 1,5-diazabicyclo[4.3.0]non-5-ene (DBN);
the first acidic thiol is selected from cysteine, thiomalic acid, and mercaptopropionic acid; and
the first coupling reagent is selected from N,N′-dicyclohexylcarbodiimide (DCC), N,N′-diisopropylcarbodiimide (DIC), N-(3-dimethylaminopropyl)-N′-ethylcarbodiimide (EDC), (benzotriazol-1-yloxy)tripyrrolidinophosphonium hexafluorophosphate (PyBOP), [ethyl cyano(hydroxyimino)acetato-O²]tri-1-pyrrolidinylphosphonium hexafluorophosphate (PyOxim), O-(benzotriazol-1-yl)-N,N,N′,N′-tetramethyluronium tetrafluoroborate (TBTU), O-(1H-6-chlorobenzotriazole-1-yl)-1,1,3,3-tetramethyluronium hexafluorophosphate (HCTU), 2-(1H-benzotriazole-1-yl)-1,1,3,3-tetramethyluronium hexafluorophosphate (HBTU), 1-hydroxybenzotriazole (HOBt), ethyl 1-hydroxy-1H-1,2,3-triazole-4-carboxylate (HOCt), 1-hydroxy-7-azabenzotriazole (HOAt), 4-dimethylaminopyridine (DMAP), and combinations of the foregoing.

12. (canceled)

13. (canceled)

14. (canceled)

15. The method of claim 1, wherein the (i) condensing, the (ii) washing, the (iii) removing, and the (iv) washing are performed in one pot.

16. The method of claim 1, further comprising repeating the (i) condensing, the (ii) washing, the (iii) removing, and the (iv) washing for one or more cycles, wherein:
the second C-protected peptide from the (iv) washing in a cycle becomes the first C-protected peptide in the (i) condensing of the following cycle; and
the first N-Fmoc amino acid or the first N-Fmoc peptide of the (i) condensing of a cycle may be the same as or different from the first N-Fmoc amino acid or the first N-Fmoc peptide of the (i) condensing of the preceding cycle.

17. The method of claim 16, wherein the (i) condensing of a cycle is performed without an intervening isolation of the second C-protected peptide from the second organic layer of the (iv) washing of the preceding cycle.

18. The method of claim 16, wherein the one or more cycles are performed in one pot.

19. The method of claim 1, further comprising:
(v) condensing an N-terminal amino group of the second C-protected peptide with a C-terminal carboxy group of a second N-Fmoc amino acid or a second N-Fmoc peptide in the presence of a second coupling reagent to obtain a third reaction mixture comprising a second N-Fmoc C-protected peptide, wherein the condensing is performed without an intervening isolation of the second C-protected peptide from the second organic layer;
(vi) washing the third reaction mixture with a third aqueous solution and separating a third organic layer comprising the second N-Fmoc C-protected peptide;
(vii) removing an N-terminal Fmoc group from the second N-Fmoc C-protected peptide to obtain a fourth reaction mixture comprising a third C-protected peptide, wherein the removing is performed without an intervening isolation of the second N-Fmoc C-protected peptide from the third organic layer;
(viii) precipitating the third C-protected peptide in a polar solvent system; and
(ix) isolating the third C-protected peptide by solid-liquid separation.

20. The method of claim 19, wherein the (v) condensing occurs in a third solvent system comprising 2-MeTHF, optionally at a concentration in the range of 70% (v/v) to 100% (v/v).

21. The method of claim 19, wherein the third solvent system further comprises dimethylformamide (DMF) or dimethylformamide (DMSO).

22. (canceled)

23. The method of claim 19, further comprising removing the lipophilic molecular anchor group from the third C-protected peptide, wherein the lipophilic molecular anchor group is a benzyl alcohol anchor group and the benzyl alcohol anchor group is removed from the third C-protected peptide by base-catalyzed ester hydrolysis.

24. (canceled)

25. The method of claim 23, wherein the base used for base-catalyzed ester hydrolysis is lithium hydroxide and optionally further wherein the base-catalyzed ester hydrolysis is performed in a solvent system comprising water and THF, optionally in a 1:4 volumetric ratio.

26. A method of producing a peptide comprising:
(i) condensing an N-terminal amino group of a first C-protected peptide with a C-terminal carboxy group of a first N-Boc amino acid or a first N-Boc peptide to obtain a first N-Boc C-protected peptide, wherein a C-terminal carboxy group of the first C-protected peptide is protected by a first benzyl alcohol anchor group;
(ii) removing the first benzyl alcohol anchor group from the first N-Boc C-protected peptide to obtain a second N-Boc peptide;
(iii) condensing a C-terminal carboxy group of the second N-Boc peptide with an N-terminal amino group of a second C-protected peptide to obtain a second N-Boc C-protected peptide, wherein a C-terminal carboxy group of the second C-protected peptide is protected by a second benzyl alcohol anchor group; and
(iv) removing the second benzyl alcohol anchor group from the second N-Boc C-protected peptide to obtain a third N-Boc peptide.

27. The method of claim 26, wherein the first benzyl alcohol anchor group is derived from 3,4,5-tri(octadecyloxy)benzyl alcohol and/or the second benzyl alcohol anchor group is derived from 3,4,5-tri(octadecyloxy)benzyl alcohol.

28. (canceled)

29. The method of claim 26, wherein:
the (ii) removing comprises removing the first benzyl alcohol anchor group from the first N-Boc C-protected peptide by base-catalyzed ester hydrolysis to obtain a first reaction mixture comprising the second N-Boc peptide; and/or
the (iv) removing comprises removing the second benzyl alcohol anchor group from the second N-Boc C-protected peptide by base-catalyzed ester hydrolysis to obtain a second reaction mixture comprising the third N-Boc peptide.

30. The method of claim 29, wherein the base used for base-catalyzed ester hydrolysis in the (ii) removing and/or the (iv) removing is lithium hydroxide, and optionally further wherein the base-catalyzed ester hydrolysis in the (ii) removing and/or the (iv) removing is performed in a solvent system comprising water and THF, optionally in a 1:4 volumetric ratio.

31. The method of claim 29, wherein, between the (ii) removing and the (iii) condensing, the method further comprises:
(iia) acidifying and diluting the first reaction mixture comprising the second N-Boc peptide;
(iib) separating a first organic layer comprising the second N-Boc peptide and concentrating the first organic layer to obtain a first concentrated mixture comprising the second N-Boc peptide;
(iic) dissolving the first concentrated mixture in a first ethereal solvent, precipitating the first benzyl alcohol anchor group in a first polar solvent system, and removing the first benzyl alcohol anchor group by solid-liquid separation to obtain a first filtrate comprising the second N-Boc peptide; and
(iid) concentrating the first filtrate to isolate the second N-Boc peptide.

32. The method of claim 26, wherein the method further comprises repeating the (iii) condensing and the (iv) removing for one or more cycles, wherein:
the third N-Boc peptide from the (iv) removing in a cycle becomes the second N-Boc peptide in the (iii) condensing of the following cycle; and
the second C-protected peptide of the (iii) condensing of a cycle may be the same as or different from the second C-protected peptide of the (iii) condensing of the preceding cycle.

33. The method of claim 32, wherein:
the (iv) removing comprises removing the second benzyl alcohol anchor group from the second N-Boc C-protected peptide by base-catalyzed ester hydrolysis to obtain a second reaction mixture comprising the third N-Boc peptide; and
between the (iv) removing of a cycle and the (iii) condensing of the following cycle, the method further comprises:
(iia) acidifying and diluting the second reaction mixture comprising the third N-Boc peptide;
(iib) separating a second organic layer comprising the third N-Boc peptide and concentrating the second organic layer to obtain a second concentrated mixture comprising the third N-Boc peptide;
(iic) dissolving the second concentrated mixture in a second ethereal solvent, precipitating the second benzyl alcohol anchor group in a second polar solvent system, and removing the second benzyl alcohol anchor group by solid-liquid separation to obtain a second filtrate comprising the third N-Boc peptide; and
(iid) concentrating the second filtrate to isolate the third N-Boc peptide.

34. The method of claim 32, wherein, following a final (iii) condensing, a final N-Boc C-protected peptide is globally deprotected to obtain a peptide that does not comprise a C-terminal protecting group, an N-terminal protecting group, and/or a side chain protecting group.
